(19) United States
(12) Patent Application Publication
(10) Pub. No.: US 2023/0411008 A1
(43) Pub. Date: Dec. 21, 2023

(54) ARTIFICIAL INTELLIGENCE AND MACHINE LEARNING TECHNIQUES USING INPUT FROM MOBILE COMPUTING DEVICES TO DIAGNOSE MEDICAL ISSUES

(71) Applicant: The Covid Detection Foundation d/b/a Virufy, San Jose, CA (US)

(72) Inventors: Amil Khanzada, San Jose, CA (US); Richard Swartzbaugh, San Jose, CA (US)

(21) Appl. No.: 18/329,461

(22) Filed: Jun. 5, 2023

Related U.S. Application Data
(60) Provisional application No. 63/348,772, filed on Jun. 3, 2022.

(51) Int. Cl. G16H 50/20; G16H 50/30

(52) U.S. Cl. CPC G16H 50/20; G16H 50/30

(57) ABSTRACT

Provided is a process, including: obtaining data from a sensor or user-interface of a mobile computing device gathered during use of the mobile computing device by a user; inferring, from the data, with a trained machine learning model, a mental-health state of the user; and storing the mental health state in memory.

### CROSS-REFERENCE TO RELATED APPLICATIONS

This patent claims the benefit of U.S. Provisional Patent Application 63/348,772, filed 3 Jun. 2022, titled CLASSIFYING AUDIO FROM LIVING ORGANISMS WITH MACHINE LEARNING. The entire content of each afore-listed earlier-filed application is hereby incorporated by reference for all purposes.

### BACKGROUND

### 1. Field

The present disclosure relates generally to artificial intelligence and machine-learning and, more specifically, to classifying mental-health states of users based on data acquired through a mobile computing device, such as a smartphone or wearable computing device.

### 2. Description of the Related Art

It can be challenging for people to know their own mental-health state. Self diagnosis is unreliable, and access to medical health professionals can be time consuming, slow, expensive, and many individuals may not have ready access to mental health support. Yet the magnitude of the effects on a person's quality of life from their mental health can be hard to overstate, not to mention the effect on the lives of those around them, their employers, and society as a whole.

### SUMMARY

The following is a non-exhaustive listing of some aspects of the present techniques. These and other aspects are described in the following disclosure.

Some aspects include a process, including: obtaining data from a sensor or user-interface of a mobile computing device gathered during use of the mobile computing device by a user; inferring, from the data, with a trained machine learning and/or artificial intelligence (AI) model, a mental-health state of the user; and storing the mental health state in memory.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 illustrates an example computing system in which mental health states may be inferred in accordance with some embodiments;

FIG. 2 illustrates a process in which mental health states may be inferred in accordance with some embodiments; and

FIG. 3 illustrates a computing device by which the above processes and systems may be implemented in some embodiments.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the fields of computer science, machine learning, artificial intelligence, and mental health. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Examples of techniques by which the above issues may be mitigated follow.

BEHAVIORAL PATTERNS AND DIGITAL BIOMARKERS: Monitoring user behavior patterns, in some embodiments, such as app usage, typing speed, scrolling behavior, or typing errors, can generate digital biomarkers indicating cognitive changes, attention deficits, or motor abnormalities associated with mental health disorders. This feature can provide valuable insights into an individual's mental health status.

Behavioral Patterns and Digital Biomarkers refer to the analysis of user behavior patterns and digital data generated through interactions with a smartphone or other digital devices. By monitoring various aspects of user behavior, in some embodiments, such as app usage, typing speed, scrolling behavior, or typing errors, it is possible to generate digital biomarkers that can provide insights into an individual's mental health status.

Once the user grants access permissions, in some embodiments, the aspects monitored below generate smartphone logs and data which can be fed in as features to various machine learning architectures (e.g., neural networks trained with deep learning, decision trees, dynamic Bayesian networks, reinforcement learning models, clustering embedding spaces, etc.) in a multimodal approach to classify patterns indicative of the proposed mental health disorders. The AI models can be trained with a baseline in normal app and device usage and determine irregularity through the severity of irregularities.

App Usage: Analyzing the types of applications a person uses, the frequency of app usage, and the duration of usage, in some embodiments, can provide insights into their daily routines, interests, and potential changes in behavior. For example, significant changes in app usage patterns, such as a decrease in productivity apps or an increase in social media usage, might indicate a shift in mental health or cognitive functioning.

Typing Speed and Errors: Monitoring voice and typing input speed and the occurrence of input errors can, in some embodiments, provide clues about cognitive changes and attention deficits. For instance, a noticeable decrease in input speed or an increase in input errors might suggest cognitive impairment or difficulties with focus and attention.

Scrolling Behavior: Analyzing scrolling and other navigation behavior, such as the speed, frequency, and direction of navigation, can, in some embodiments, provide information about engagement levels, cognitive processing, and potential emotional states. Unusual navigation patterns, such as repetitive or erratic navigation, could indicate heightened anxiety or compulsive behaviors.

Digital Distractions: Monitoring the frequency and duration of interruptions or distractions during digital tasks can, in some embodiments, provide insights into attention deficits and difficulties with concentration. Excessive interruptions or switching between tasks may indicate challenges in sustaining attention, which is often associated with certain mental health conditions.

Sleep Patterns: Analyzing patterns of digital device usage during different times of the day or night can, in some embodiments, provide information about sleep patterns, disturbances or disruptions. Irregular sleep patterns, such as excessive nighttime device usage or disrupted sleep-wake cycles, may indicate sleep disorders or mood-related disturbances.

Digital Footprint: Examining an individual's digital footprint, including their online interactions, social media activity, and browsing history, can, in some embodiments, provide additional contextual information about their mental health. For example, changes in language use, social withdrawal patterns, or increased engagement with specific topics or communities might indicate changes in mood or mental well-being. Additionally, analysis of reactions and responses to user posted content, where device and app permissions permit, can also be analyzed similarly.

FACIAL AND BODY MOVEMENT EXPRESSION/EMOTION RECOGNITION: Analyzing facial expressions captured by the smartphone's camera and using machine learning algorithms to recognize emotions in real-time (e.g., within less than 5 seconds of acquiring an input) or in a delayed fashion can, in some embodiments, assist in assessing emotional states and mental health disorders. This feature can be particularly useful in detecting affective disorders, psychotic disorders, or autism spectrum disorders.

Facial Expression/Emotion Recognition involves analyzing facial expressions captured by the smartphone's camera and using machine learning algorithms (a term used broadly to also include artificial intelligence algorithms) to recognize and categorize emotions in real-time. This feature has, in some embodiments, the potential to assist in assessing emotional states and identifying mental health disorders.

Facial Expression Analysis: The smartphone's camera captures facial expressions, which are then analyzed using advanced machine learning algorithms, in some embodiments. These algorithms can, in some embodiments, identify facial landmarks, movements, and patterns associated with different emotions, such as happiness, sadness, anger, fear, disgust, or surprise. Given the presence of large facial expression datasets, deep learning models can be trained to obtain automated feature extraction for relevant features corresponding facial expression changes with emotional changes.

Real-Time Emotion Recognition: By leveraging machine learning models trained on datasets of facial expressions (e.g., more than 500 labeled example images), the smartphone can, in some embodiments, provide real-time analysis of emotional states. This means that as the person's face is being recorded, the algorithm can, in some embodiments, quickly analyze and recognize the displayed emotions through the use of deep learning and multimodal models, e.g merging information obtained from analysis of joint movement or skin color variation such as redness while angry.

Assessing Emotional States: Facial expression analysis can, in some embodiments, help assess an individual's emotional states and fluctuations over time. It can provide objective information about the intensity and duration of specific emotions, helping to understand an individual's emotional well-being.

Detection of Mental Health Disorders: Facial expression analysis can, in some embodiments, also be used to detect certain mental health disorders. For example, affective disorders like depression and anxiety are often characterized by distinct facial expression patterns. Similarly, psychotic disorders, such as schizophrenia, may exhibit specific abnormalities in facial expressions that can be detected through this technology. Additionally, facial expression analysis has shown promise in assisting with the early detection of autism spectrum disorders by identifying atypical facial expressions associated with social communication difficulties.

Non-invasive and Convenient: Facial expression analysis using a smartphone's camera is, in some embodiments, a convenient method for capturing emotional cues. It eliminates the need for additional sensors or equipment, making it easily accessible and feasible for widespread use.

Potential Limitations: While facial expression analysis has significant potential, there are some limitations to consider. It may not capture subtle or nuanced emotional expressions accurately, and individual variations in facial expressions may affect the accuracy of emotion recognition. Cultural differences in facial expressions, differences in skin tones/facial structure, and privacy concerns regarding facial data should also be taken into account. Additionally, true emotions may not be detectable from faked facial expressions.

CONTEXTUAL MOOD TRACKING: Prompting users to provide self-reported or automatically inferred mood ratings throughout the day based on their current activities, locations, or social interactions can help identify patterns and triggers associated with specific mental health symptoms or conditions. This feature can, in some embodiments, provide contextual information for assessing mental health.

Contextual Mood Tracking, in some embodiments, involves prompting users to provide self-reported or automatically inferred mood ratings at different times throughout the day, taking into account their current activities, locations, or social interactions. This feature aims to identify patterns and triggers associated with specific mental health symptoms or conditions. Here are more details about this capability:

Self-Reported Mood Ratings: Users, in some embodiments, are prompted to provide subjective mood ratings using predefined scales or descriptors. These ratings typically reflect the user's current emotional state, such as happiness, sadness, anxiety, or stress. Users may be asked to rate their mood multiple times a day or at specific intervals.

Automatic Inference of Mood: In addition to self-reported mood ratings, contextual mood tracking can, in some embodiments, also leverage other data sources to automatically infer the user's mood. This can, in some embodiments, include information such as the user's location, activity level, social interactions, social media app interactions, typing speed, phone usage frequency, or physiological measurements like heart rate or sleep quality. Machine learning algorithms can be employed to analyze this data and estimate the user's mood without explicit input.

Contextual Information: Contextual mood tracking, in some embodiments, takes into account various factors that may influence a person's mood, including their current activities, locations, and social interactions. For example, a user may be asked to rate their mood after a work meeting, during a social gathering, or while engaging in a specific activity like exercise. By linking mood ratings to these contextual cues, in some embodiments, patterns and triggers associated with specific mental health symptoms or conditions can be identified.

Identifying Patterns and Triggers: By collecting and analyzing mood ratings in conjunction with contextual information, in some embodiments, patterns and triggers associated with specific mental health symptoms or conditions can be identified. For instance, the data may reveal that a person experiences increased anxiety during work-related tasks or improved mood after engaging in physical exercise. This information can, in some embodiments, help individuals, as well as mental health professionals, gain insights into the factors influencing their mental well-being.

Personalized Insights and Interventions: Contextual mood tracking can enable the development of personalized insights and interventions. By understanding the specific triggers and patterns affecting an individual's mood, tailored recommendations and interventions can be provided. For example, if a person consistently experiences low mood after certain activities, they may be encouraged to engage in alternative or coping strategies to improve their well-being.

Longitudinal Tracking: Contextual mood tracking, in some embodiments, is most effective when implemented longitudinally, capturing data over an extended period. This allows for the identification of trends and changes in mood patterns over time, providing a more comprehensive understanding of an individual's mental health.

CONTEXTUAL SENSOR FUSION: Combining data from multiple sensors, such as accelerometer, GPS, microphone, and heart rate sensor, can, in some embodiments, offer a comprehensive view of an individual's daily activities, social interactions, physiological responses, and environmental factors. This contextual sensor fusion can, in some embodiments, provide comprehensive insights into mental health and help identify patterns and correlations.

Contextual Sensor Fusion, in some embodiments, involves combining data from multiple sensors, such as accelerometer, GPS, microphone, and heart rate sensor, to offer a comprehensive view of an individual's daily activities, social interactions, physiological responses, and environmental factors. This fusion of sensor data can, in some embodiments, provide comprehensive insights into mental health and help identify patterns and correlations.

1. Multiple Sensor Inputs: Contextual sensor fusion, in some embodiments, integrates data from various sensors that are commonly found in smartphones or wearable devices. These sensors may include:

a. Accelerometer: Measures the device's acceleration and movement, providing information about physical activity, gestures, and behavior patterns, in some embodiments.

b. GPS (or other geolocation sensing, like cell-tower triangulation, or other satellite navigation services): Tracks, in some embodiments, the user's location and movement, allowing for analysis of mobility patterns, travel habits, and exposure to different environments.

c. Microphone: Records, in some embodiments, audio data, capturing speech patterns, social interactions, and ambient sounds.

d. Heart Rate Sensor: Measures, in some embodiments, the user's heart rate and heart rate variability, providing insights into physiological arousal, stress levels, and emotional states.

2. Comprehensive View of Activities and Interactions: By combining data from multiple sensors, contextual sensor fusion, in some embodiments, offers a comprehensive view of an individual's daily activities and social interactions. For example, it can provide information on the user's physical activity levels throughout the day, their location history, the duration and frequency of social interactions, and even the tone of their conversations in person and through digital mediums.

3. Physiological and Environmental Factors: Contextual sensor fusion, in some embodiments, incorporates physiological and environmental data to understand their impact on mental health. For instance, it can analyze the user's heart rate patterns in different situations, such as during exercise, sleep, or stressful events. It can, in some embodiments, also consider environmental factors like noise levels or air quality, which can influence mood and well-being.

4. Identifying Patterns and Correlations: By fusing data from multiple sensors, in some embodiments, patterns and correlations can be identified. For example, the data may reveal that the user experiences higher stress levels when engaged in certain activities or during specific times of the day. It may also show associations between physiological responses, such as increased heart rate, and environmental factors like noise levels. These insights can, in some embodiments, help individuals and mental health professionals understand the factors that contribute to mental well-being or potential triggers for mental health conditions.

5. Personalized Interventions: Contextual sensor fusion, in some embodiments, facilitates the development of personalized interventions based on the comprehensive data collected. By understanding the relationship between sensor data and mental health outcomes, in some embodiments, tailored recommendations can be provided. For instance, if the data indicates that a person experiences increased stress levels during specific activities, they may be encouraged to practice relaxation techniques or modify their routines to manage stress more effectively.

6. Privacy and Data Security: Since contextual sensor fusion involves collecting and analyzing sensitive data, in some embodiments, privacy and data security are of paramount importance. Appropriate measures should be taken to ensure the secure handling and storage of user data, including anonymization and encryption techniques, e.g. through use of synthetic generative AI such as with generative adversarial networks (GANs) to translate personally identifiable user data into an anonymized form which appears like real data and is useable for AI model training. Users should have control over their data and the option to provide informed consent before sharing it.

MACHINE LEARNING ALGORITHMS: Advanced machine learning algorithms can, in some embodiments, analyze various sensor data collected from the cell phone to identify patterns, detect anomalies, and predict mental health conditions. These algorithms can, in some embodiments, integrate multiple data sources and provide personalized assessments, making them a powerful tool for mental health diagnosis.

Machine learning algorithms, in some embodiments, play a role in analyzing the various sensor data collected from smart phones and other devices to identify patterns, detect anomalies, and predict mental health conditions. These algorithms, in some embodiments, leverage the power of artificial intelligence to process large amounts of data and derive meaningful insights.

1. Data Analysis and Pattern Recognition: Machine learning algorithms can, in some embodiments, analyze the collected sensor data, including behavioral patterns, facial expressions, mood ratings, and contextual information, to identify patterns and correlations. By learning from a large dataset, these algorithms can recognize complex patterns that might be difficult for humans to detect. For example, they can, in some embodiments, identify specific app usage patterns associated with mental health disorders or detect subtle changes in facial expressions indicative of emotional states.

2. Integration of Multiple Data Sources: Machine learning algorithms, in some embodiments, are capable of integrating data from multiple sources, such as accelerometer, GPS, microphone, and heart rate sensor, to gain a comprehensive understanding of an individual's mental health. By considering various data streams simultaneously, these algorithms can, in some embodiments, provide a more accurate and holistic assessment of mental well-being. For instance, they can combine data on physical activity levels, location history, and social interactions to infer the impact of social support networks on mental health.

3. Personalized Assessments and Predictions: Machine learning algorithms can, in some embodiments, generate personalized assessments and predictions based on the collected data. These algorithms can, in some embodiments, learn from historical data to develop models that are specific to an individual's characteristics, device usage patterns, and mental health profile. By considering individual differences and unique patterns, the algorithms can, in some embodiments, provide tailored insights and predictions. For example, they can predict the likelihood of a depressive episode based on past behavioral patterns and contextual information.

4. Continuous Learning and Adaptation: Machine learning algorithms, and artificial intelligence, in some embodiments, have the ability to continuously learn and adapt as new data becomes available. This dynamic learning process, in some embodiments, allows the algorithms to improve their accuracy and effectiveness over time. As more data is collected and new patterns are discovered, the algorithms can, in some embodiments, update their models and predictions, leading to more refined assessments of mental health conditions, with active learning.

5. Ethical Considerations: When developing and deploying machine learning algorithms for mental health assessment, ethical considerations are paramount. It is crucial to ensure the algorithms are trained on diverse and representative datasets to avoid biases and ensure fair and accurate predictions. Privacy and data security measures should be in place to protect individuals' sensitive information. Additionally, transparency and explainability of the algorithms are important, enabling users and mental health professionals to understand how the predictions are generated and the factors considered.

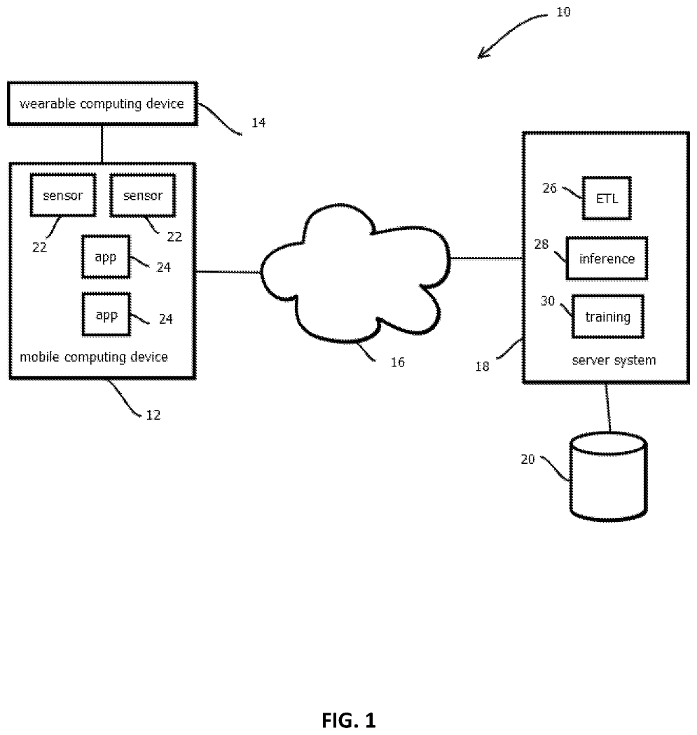

FIG. 1 illustrates an example computing system 10 that may implement techniques like those described above in some embodiments. Some embodiments include a mobile computing device 12 and a wearable computing device 14 that communicate, via the Internet 16, to a server or cloud-based system 18 with access to a training set 20. The illustrated system 10 implements a client-server architecture, but contemplated embodiments include other architectures, including monolithic systems implemented solely on the mobile computing device 12.

In some embodiments, the mobile computing device 12 includes an operating system in which a plurality of native applications 24 installed on the mobile computing device 12 execute. Some of these applications may be productivity applications, social media applications, games, and the like, each of which may be categorized in a taxonomy of such applications including categories like those mentioned. In some embodiments, the applications 24 include an application configured to communicate with the server system 18 and implement the processes described herein.

In some embodiments, the mobile computing device 12 is a cell phone, smartphone, a tablet computer, a laptop computer, a head-mounted computer (or other wearable computing device), and in some embodiments, the mobile computing device 12 communicates with wearable computing devices 14 worn by the user and configured to supplement the sensor suite of the mobile computing device 12. The mobile computing device 12 may receive sensor data from local drones, portable cameras, microphones, and other input devices, in some embodiments. In some cases, the wearable device 14 may be a surgically embedded device, like a pacemaker, a cochlear implant, a brain-computer interface, an embedded radio frequency identifier or ultra-wideband device, or the like. The data described as gathered by device 12 may be initially gathered by device 14. In some embodiments, the mobile computing device 12 includes a plurality of sensors 22, examples of which are described above. The mobile computing device 12 may further include various cellular radios, Bluetooth™, NFC (near field communication), ultra-wideband radios, or Wi-Fi™ radios by which the mobile computing device 12 communicates via the Internet 16.

In some embodiments, the server system 18 includes an extract transform and load (ETL) module 26, an inference module 28, and a training module 30. In some embodiments, the ETL module 26 may receive data from the mobile computing device 12, cleanse the data by filtering noise and in some cases implement engineered features that serve as inputs to a trained model executed by the inference module 28. The machine learning module executed at runtime by inference module 28 may include a plurality of sub-models, for example in an ensemble or in a pipeline, and in some embodiments, these models may be trained independently or with end-to-end training using the training module 30 based upon training data in the initial training data set 20. As noted, models may be run at interference time on the mobile computing device 12 or the server system 18, depending on the use case. Depending on the model type, training may be supervised, unsupervised, or semi-supervised. In some embodiments, the functionality of the server system 18 may be executed client side by the mobile computing device 12 in whole or in part. In some embodiments, the server system (which may be an on-premises or cloud-based system) 18 may interface with the plurality of mobile computing devices of a plurality of different users, such as more than 1000, more than 100,000, or more than a million users geographically distributed over the entire country or the globe, accounting for global factors affecting large populations of users in parallel (e.g., wars, pandemics, presidential elections).

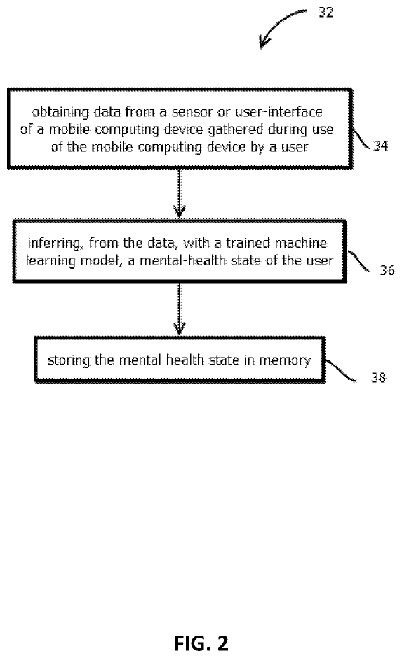

In some embodiments, the system 10 above or other architectures may execute a process 32 illustrated by FIG. 2. These steps of the process 32 may be executed in a different order from that shown, additional steps may be inserted, some steps may be omitted, some steps may be executed concurrently or serially, and some steps may be repeated, none of which is to suggest that other aspects describe herein are not also subject to modification. In some embodiments, the process 32 may be embodied by machine readable instructions stored on a tangible, non-transitory, machine readable medium, such that when the instructions are executed the described functionality is implemented.

Some embodiments implement a process 30 shown in FIG. 2 that includes obtaining data from a sensor or user-interface of a mobile computing device gathered during use of the mobile computing device by a user, as indicated by block 34. Some embodiments may further include inferring from the data, with a trained machine learning model (such as an artificial intelligence model), a mental-health state of the user, as indicated by block 36. Embodiments further include storing the mental health state of the user in memory, as indicated by block 38. Embodiments may include making the above-described personalized recommendations to users or providing other types of feedback described above and training the model. Embodiments may include incremental learning of the model by incorporating new training data from the current user and global users.

Other examples that may be implemented with the system or process above follow.

It should be emphasized that all examples herein are prophetic.

Examples of diagnosing mental health disorders using features and functions of a smartphone such as the camera, microphone, accelerometer, GPS (a term used herein to also refer to other techniques by which geolocation is sensed beyond and including satellite navigation), gyroscope (or inertial measurement unit with gyros and accelerometers, such as a three or six axis IMU), magnetometer (compass), proximity sensor, ambient light sensor, barometer, thermometer, heart rate sensor, fingerprint sensor, electrodermal, electric nose, perspiration, temperature, facial recognition sensor, camera, touchscreen.

1. Accelerometer and Gyroscope: These sensors can detect motion and orientation of the smartphone. By analyzing movement patterns, cadence, human locomotion, fall risk, gait, or tremors, they can provide insights into motor-related disorders, such as Parkinson's disease or restless leg syndrome.

2. Accelerometer, Gyroscope, and Magnetometer (Compass): These sensors can track movement, posture, and orientation. They can be used to analyze physical activity levels, sleep patterns, and motor abnormalities associated with certain mental health disorders.

3. Ambient Light Sensor: The ambient light sensor can measure changes in the surrounding light levels. It can be used to gather information about sleep/wake patterns or light exposure, which can be relevant to mood disorders.

4. Ambient Noise Sensor: The ambient noise sensor can analyze the surrounding sound levels and patterns. It can be used to detect changes in the acoustic environment, such as increased noise exposure or changes in speech patterns, which may be relevant to mental health conditions like auditory hallucinations or social anxiety disorder.

5. Barometer: The barometer measures atmospheric pressure. Changes in atmospheric pressure can potentially be linked to migraines or other mental health conditions that are influenced by environmental factors. In addition to measuring atmospheric pressure, it can also detect changes in altitude. It can be utilized to analyze associations between altitude changes and mood disorders, such as bipolar disorder.

6. Behavioral Patterns and Digital Biomarkers: By monitoring user behavior patterns, such as app usage, typing speed, scrolling behavior, or typing errors, smartphones can generate digital biomarkers that indicate cognitive changes, attention deficits, or motor abnormalities associated with mental health disorders.

7. Biometric Authentication Analysis: The smartphone's fingerprint sensor or facial recognition sensor can be used to analyze biometric data during specific tasks or interactions. Changes in biometric measures, such as heart rate, skin conductance, or facial expressions, can indicate emotional states or stress levels.

8. Biometric Data Fusion: Integrating data from multiple sensors, such as heart rate, movement, and environmental factors, can create a more comprehensive picture of the user's mental health state. These data fusion techniques enable a more holistic assessment and personalized monitoring.

9. Camera: The camera can be used for facial or bodily expression analysis to detect emotional states, signs of anxiety or depression, or other facial cues associated with mental health disorders, which may also include non-mental health disorders including stroke.

10. Cognitive Assessment Apps: Using the touchscreen and accelerometer, cognitive assessment apps can deliver tasks and exercises to evaluate cognitive functions such as memory, attention, problem-solving, or executive function. These assessments can aid in the diagnosis of conditions like dementia, attention deficit hyperactivity disorder (ADHD), or cognitive impairment.

11. Contextual Environmental Triggers: By leveraging data from the smartphone's sensors, including GPS, accelerometer, and ambient light sensor, contextual environmental triggers can be identified. These triggers can include specific locations, activities, or environmental conditions that exacerbate or alleviate mental health symptoms.

12. Contextual Mood Tracking: The smartphone can prompt users to provide self-reported or automatically inferred (e.g., through use of AI voice analytics) mood ratings throughout the day based on their current activities, locations, or social interactions. This contextual mood tracking can help identify patterns and triggers associated with specific mental health symptoms or conditions.

13. Contextual Sensor Fusion: By combining data from multiple sensors, such as accelerometer, GPS, microphone, and heart rate sensor, the cell phone can provide a holistic view of an individual's daily activities, social interactions, physiological responses, and environmental factors. This contextual sensor fusion can offer comprehensive insights into mental health and help identify patterns and correlations.

14. Data Visualization and Insights: smartphone applications can provide users with visual representations of their mental health data, including mood trends, stress levels, sleep patterns, and activity levels. This allows individuals to gain a better understanding of their mental well-being and make informed decisions about their mental health.

15. Digital Social Interaction Analysis: By analyzing data from social media platforms, text messages, or call logs, patterns of social interaction can be studied. Changes in communication patterns, social withdrawal, or uncharacteristic language use can be indicative of changes in mood and mental and/or emotional states.

16. Electrodermal Activity (EDA): By measuring changes in skin conductance or perspiration levels, the electrodermal activity sensor can provide information about emotional arousal, stress, or anxiety responses.

17. Electrodermal Activity Sensor: This sensor measures the electrical conductivity of the skin, which can provide insights into emotional arousal and stress levels.

18. Facial Expression/Emotion Recognition: By analyzing facial expressions captured by the cell phone's camera, combined with machine learning algorithms, the device can recognize and categorize emotions in real-time. This can assist in assessing emotional states and mental health disorders, such as affective disorders (depression, anxiety), psychotic disorders, or autism spectrum disorders.

19. Environmental Context Detection: Utilizing the smartphone's sensors, including GPS, accelerometer, and ambient light sensor, the device can detect contextual information such as location, activity level, and exposure to environmental factors. This data can be integrated with mental health assessments to understand the impact of the environment on mental well-being.

20. Environmental Noise Analysis: The smartphone's microphone can analyze environmental noise levels and patterns. Excessive noise exposure or sensitivity to certain sounds can be associated with mental health conditions such as anxiety, sensory processing disorders, or post-traumatic stress disorder (PTSD).

21. Facial Expression Analysis: Utilizing the camera, facial recognition sensor, and machine learning algorithms, the smartphone can analyze facial expressions to detect signs of emotional distress, such as sadness, anger, shock, or anxiety. This can provide insights into mood disorders and assist in early detection.

22. Facial Recognition Sensor: Facial recognition technology can analyze facial features, expressions, and micro expressions to detect emotional states, signs of depression, or other mental health-related cues, and non-mental health including stroke.

23. Fingerprint Sensor: The fingerprint sensor can be used to identify stress-related changes in perspiration patterns and potentially indicate anxiety or other mental health conditions.

24. Gamified Assessments: Leveraging the touchscreen, accelerometer, and other sensors, the smartphone can deliver interactive and engaging assessments that incorporate game-like elements. These assessments can measure cognitive function, attention, memory, or emotional responses in a more enjoyable and user-friendly manner.

25. Geolocation and Activity Tracking: By utilizing the GPS and accelerometer sensors, the smartphone can track a user's location, movement, and activity levels. Changes in mobility or engagement in daily activities can provide indicators of mental health disorders such as depression or bipolar disorder, or the state of mania.

26. GPS (Global Positioning System): GPS technology can track the user's location and movement patterns. It can provide insights into behavioral patterns, such as agoraphobia or social anxiety disorder, by analyzing avoidance behaviors or identifying triggering locations.

27. Heart Rate Sensor: The heart rate sensor measures the user's heart rate and can provide information about their physiological arousal and stress levels.

28. Heart Rate Variability (HRV): The heart rate sensor can measure HRV, which reflects the variability in time intervals between heartbeats. HRV analysis can indicate autonomic nervous system dysregulation and provide insights into stress, anxiety, or mood disorders.

29. Light Exposure Sensor: This sensor can monitor the user's exposure to different levels of light, including natural and artificial light sources. It can provide information about circadian rhythms, sleep disturbances, or seasonal affective disorder.

30. Machine Learning Algorithms: Advanced machine learning algorithms can analyze various sensor data collected from the cell phone to identify patterns, detect anomalies, and predict mental health conditions. These algorithms can integrate multiple data sources and provide personalized assessments.

31. Microphone: The microphone can capture voice recordings for speech, cough, breathing, wheezing, sneezing, or other respiratory analysis, detecting changes in audio patterns, tone, and speech rate that may indicate mental health or respiratory symptoms.

32. Mood Prediction Models: By collecting data from various sensors, including the camera, microphone, accelerometer, and heart rate sensor, machine learning algorithms, and artificial intelligence can analyze patterns and physiological indicators to predict mood fluctuations and episodes related to mental health disorders.

33. Personalized Digital Interventions: Using the touchscreen and app-based interfaces, smartphones can deliver personalized digital interventions such as cognitive-behavioral therapy (CBT) exercises, mindfulness practices, relaxation techniques, or mood regulation strategies. These interventions can be tailored based on individual needs and preferences, promoting self-management and well-being, individually or in conjunction with therapists to better manage therapy assignments throughout the week.

34. Perspiration Sensor: This sensor can measure sweat production and can be used to monitor changes in perspiration levels, which may indicate stress, anxiety, or panic responses.

35. Physiological Response Monitoring: By integrating with wearable devices or external sensors, such as heart rate monitors or electrodermal activity sensors, the cell phone can capture physiological responses in real-time. These responses can be used to detect stress levels, anxiety, or emotional arousal, providing insights into mental health states.

36. Proximity Sensor: The proximity sensor can detect the distance between the phone and the user's face. It can be utilized to measure physiological responses like increased proximity during anxious or stressful situations.

37. Sleep Monitoring: The accelerometer and ambient light sensor, along with battery charging timings, can be utilized to track sleep patterns, including sleep duration, quality, and disturbances. Changes in sleep patterns can provide insights into various mental health disorders, such as insomnia, depression, or bipolar disorder.

38. Social Media Analysis: By integrating with social media platforms, the cell phone can analyze user activity, language patterns, and social interactions to assess mental health states. This can include sentiment analysis, identifying signs of social withdrawal or excessive social media use, and detecting cyberbullying or harassment.

39. Social Network Analysis: By analyzing data from the cell phone's contact list, call logs, and messaging apps, social network analysis can provide insights into an individual's social connections and support systems. Changes in social interaction patterns or the quality of relationships can be indicative of mental health symptoms.

40. Speech and Language Analysis: The cell phone's microphone and natural language processing algorithms can analyze speech patterns, language use, and word choice to detect signs of mental health disorders such as schizophrenia, dementia, or cognitive impairment. Changes in speech characteristics can provide valuable diagnostic information.

41. Temperature Sensor: The temperature sensor can measure the user's skin temperature, which can be influenced by emotional and psychological states. Changes in temperature patterns can provide insights into stress levels or emotional regulation.

42. Thermometer: The thermometer can measure the ambient temperature. Fluctuations in temperature may be associated with stress responses or changes in mood.

43. Interaction Analysis: By analyzing touchscreen interaction patterns, such as typing speed, pressure, or gesture movements, changes in motor behavior or cognitive functioning can be identified. This can be useful in assessing conditions like obsessive-compulsive disorder or cognitive impairment.

44. Touchscreen: The touchscreen can be used to collect self-reported data through interactive surveys or mood tracking apps, providing valuable information about the user's mental state over time.

45. User Behavior Tracking: By monitoring app usage, browsing habits, or typing patterns, changes in behavior or deviations from the user's baseline can be detected, potentially indicating mental health issues or cognitive changes.

46. Virtual Reality (VR) Integration: By coupling the cell phone with VR technology, immersive environments can be created to simulate positive visualization exercises or therapist-approved therapeutic interventions. VR can be utilized in exposure therapy for anxiety disorders or in assessing phobias under supervision by a licensed mental health provider.

47. Virtual Reality Exposure Therapy: Utilizing the smartphone's display and motion sensors, virtual reality (VR) applications can provide immersive environments for exposure therapy. This can be particularly effective for anxiety disorders, phobias, or post-traumatic stress disorder (PTSD), allowing individuals to confront and gradually overcome their fears in a controlled virtual environment under the supervision of a licensed mental health provider.

48. Voice Analysis: The smartphone's microphone can be used to analyze vocal characteristics, such as pitch, tone, and speech patterns, to detect signs of mental health disorders like depression, schizophrenia, or bipolar disorder. Voice-based algorithms can identify patterns associated with mood changes or symptom severity. The microphone can also capture and analyze the user's voice patterns, tone, and speech characteristics. Changes in voice patterns, such as increased speech rate, vocal intensity, or abnormal prosody, can indicate symptoms of conditions like mania, depression, or schizophrenia.

49. Smell Sensor (Electric Nose): Future smartphone's may be equipped with odor sensors, will allow analysis of smells in the environment around the user or emitted by the user (e.g., alcohol smell or body odor). These can be tracked for monitoring or informing environmental impacts on user mental health, or indicators that the user may be close to an exacerbation.

50. Hygrometer: By detecting changes in air humidity, the smartphone can infer various conditions, such as aquaphobia, drowning, and water-triggered mental conditions.

Examples of diagnosing mental health disorders using features and functions of a smartphone such as the camera, microphone, accelerometer, GPS, gyroscope, magnetometer (compass), proximity sensor, ambient light sensor, barometer, thermometer, heart rate sensor, fingerprint sensor, electrodermal, perspiration, temperature, facial recognition sensor, camera, touchscreen, electrice nose, and hygrometer.

1. DSM (The DSM-5, or the Diagnostic and Statistical Manual of Mental Disorders, Fifth Edition, is a widely used diagnostic tool published by the American Psychiatric Association (APA). It provides a common language and standard criteria for the classification and diagnosis of mental disorders. The DSM-5 includes a comprehensive list of mental disorders, along with diagnostic criteria and guidelines for each disorder. These criteria help clinicians in making accurate and consistent diagnoses based on the symptoms and impairment experienced by individuals).

2. MMPI (MMPI stands for Minnesota Multiphasic Personality Inventory. It is a psychological assessment tool used to measure personality traits, psychopathology, and mental health conditions. The MMPI is one of the most widely used and researched self-report inventories for assessing adult psychopathology. The MMPI consists of a large number of questions or statements that the individual taking the test must respond to. These statements cover a wide range of topics and behaviors. The responses are typically given on a Likert scale, where the individual rates their level of agreement or disagreement with each statement. The original MMPI was developed in the 1940s and has since undergone several revisions, with the most recent version being the MMPI-2-RF (Restructured Form). The MMPI-2-RF consists of various scales and subscales that measure different aspects of personality and psychopathology, including depression, anxiety, social introversion, paranoia, and more. The MMPI-2-RF is used in clinical settings, research, and forensic evaluations to assess and diagnose various mental health disorders, evaluate treatment outcomes, and assist in treatment planning. It provides valuable information about an individual's psychological functioning and can aid in identifying patterns of behavior and symptoms associated with different mental health conditions.

3. ICD-10 (International Classification of Diseases, 10th Revision): Developed by the World Health Organization (WHO), the ICD-10 is a widely used diagnostic classification system for various medical conditions, including mental and behavioral disorders. It provides diagnostic criteria and codes for different psychiatric disorders and is often used in conjunction with the DSM-5.

4. Research Domain Criteria (RDoC): RDoC is an initiative by the National Institute of Mental Health (NIMH) that aims to understand mental disorders based on dimensions of neurobiology, behavior, and cognition rather than traditional diagnostic categories. It focuses on studying specific domains of functioning and their underlying mechanisms.

5. Structured Clinical Interview for DSM Disorders (SCID): The SCID is a semi-structured interview designed to assess and diagnose psychiatric disorders based on the DSM criteria. It provides a standardized approach to gathering information about symptoms, duration, and impairment associated with various disorders.

6. Mini International Neuropsychiatric Interview (MINI): The MINI is a brief, structured interview that assesses a range of psychiatric disorders based on both DSM and ICD criteria. It is often used in research and clinical settings as a screening tool to determine the presence or absence of specific disorders.

7. Beck Depression Inventory (BDI): The BDI is a self-report questionnaire designed to measure the severity of depressive symptoms. It assesses various aspects of depression, such as mood, guilt, appetite, and sleep disturbances, providing a quantitative measure of symptom severity.

8. Hamilton Rating Scale for Anxiety (HAM-A): The HAM-A is a clinician-administered scale that assesses the severity of anxiety symptoms. It evaluates different domains of anxiety, including psychological, somatic, and autonomic symptoms, to provide a comprehensive assessment of anxiety disorders.

9. Yale-Brown Obsessive-Compulsive Scale (Y-BOCS): The Y-BOCS is a clinician-rated scale commonly used to assess the severity of obsessive-compulsive disorder (OCD). It measures the frequency and intensity of obsessions and compulsions, as well as the interference caused by these symptoms.

10. Brief Psychiatric Rating Scale (BPRS): The BPRS is a clinician-administered scale used to assess the severity of psychiatric symptoms across multiple domains, such as positive and negative symptoms, mood, and cognitive functioning. It is often employed in research and clinical trials.

11. Brief Psychiatric Rating Scale (BPRS): The CBCL is a parent-reported questionnaire that assesses emotional and behavioral problems in children and adolescents. It covers a wide range of psychological symptoms and provides information about internalizing and externalizing problems.

Examples relevant to Diagnostic and Statistical Manual of Mental Disorders (DSM):

1. Activity Monitoring: The accelerometer and GPS features of a smartphone can be used to monitor an individual's daily activities, movement patterns, and physical behavior. By collecting data on sleep patterns, waking patterns, activity levels, mobility, and social interactions, it becomes feasible to detect abnormalities or changes that might be related to a mental health condition.

2. Audio-based Analysis: The microphone on a smartphone can be used to capture and analyze speech patterns, vocal tone, and speech characteristics. Novel approaches could involve using voice analysis algorithms to detect signs of depression, anxiety, or other mental health disorders based on speech patterns and voice modulation.

3. Behavioral Pattern Monitoring: smartphones can track various behavioral patterns, such as sleep duration, physical activity levels, social interactions, and daily routines. Analyzing these patterns in relation to DSM-5 criteria can provide insights into the presence and severity of mental health symptoms, as disruptions or deviations from normal patterns may indicate underlying mental health symptomatology.

4. Biometric Data Integration: smartphones are equipped with various sensors that can capture biometric data, including heart rate, skin conductance, and sleep patterns. Integrating this data with self-reported symptoms and DSM-5 criteria can help identify correlations between physiological markers and mental health symptoms.

5. Biometric Monitoring: smartphones equipped with biometric sensors, such as heart rate monitors or electrodermal activity sensors, can collect physiological data that may correlate with mental health disorders. By comparing these biometric measures to DSM-5 criteria, it may be possible to identify patterns and associations that aid in diagnosis.

6. Cognitive Assessment Games: Mobile applications can leverage gamification techniques to create interactive cognitive assessment tools. By combining cognitive tests aligned with DSM-5 criteria with engaging gameplay elements, these apps can provide a convenient and engaging way to assess cognitive functioning, memory, attention, and other relevant factors related to mental health disorders.

7. Contextual Data Integration: smartphones can collect various contextual data, such as location, time of day, weather conditions, and social context. By integrating this information with self-reported symptoms or behavioral data, it is possible to gain a better understanding of how environmental factors interact with mental health symptoms as defined by the DSM-5.

8. Contextual Data Integration: The smartphone can collect contextual data such as location, time, and environmental factors. By integrating this data with self-reported information and diagnostic criteria from the DSM-5, it becomes possible to gain a deeper understanding of how specific contexts or environmental factors may influence the manifestation and severity of mental health symptoms.

9. Contextualized Self-Reporting: smartphones can facilitate the collection of self-reported data through mobile applications. Innovative approaches could include context-aware prompts that trigger self-reporting based on specific situations or environmental cues. This enables the capture of real-time information aligned with DSM-5 criteria, improving the accuracy and reliability of self-reported symptoms.

10. Ecological Momentary Assessment (EMA): Using the smartphone, individuals can engage in real-time data collection and reporting of their thoughts, emotions, and behaviors. EMA techniques allow for the assessment of symptoms and experiences as they occur in daily life, providing more accurate and immediate data for diagnostic purposes.

11. Facial Expression Analysis: The camera on a cell phone can capture facial expressions, which can be analyzed using computer vision techniques. Facial expression analysis algorithms can detect and analyze emotional expressions, providing objective data to assess emotional states aligned with DSM-5 criteria.

12. Facial Expression Recognition: The camera on a smartphone can be utilized for facial expression analysis. By employing advanced computer vision techniques and machine learning algorithms, it becomes possible to identify and analyze facial expressions associated with different mental health conditions, such as depression, bipolar disorder, or post-traumatic stress disorder (PTSD).

13. Location-Based Mood Assessment: By utilizing the GPS feature of a smartphone, individuals can report their mood in real-time based on their location. This data can be combined with DSM-5 criteria to identify correlations between specific environments or situations and mental health symptoms, aiding in understanding triggers or exacerbating factors.

14. Machine Learning-Based Approaches: By combining the features of a cell phone with machine learning algorithms, it becomes possible to develop personalized diagnostic models. These models can continuously learn and adapt based on user data, enabling early detection and personalized interventions for mental health disorders based on the DSM-5 criteria.

15. Machine Learning-Based Diagnostics: Cell phones can leverage machine learning algorithms to analyze a wide range of data collected from various sensors and sources. By training models on large datasets that include DSM-5 diagnoses, it may be possible to develop predictive models that can assist in diagnosing mental health disorders based on real-time and other data captured by the cell phone.

16. Machine Learning-Enabled Symptom Recognition: By leveraging machine learning algorithms, cell phones can analyze a range of data inputs, including self-reported symptoms, voice recordings, facial expressions, activity patterns, and contextual information, to develop models that recognize patterns indicative of specific mental health disorders outlined in the DSM-5.

17. Personalized Symptom Tracking: Mobile applications can be designed to allow individuals to track their symptoms over time, capturing information aligned with the DSM-5 criteria. Through user input, such as self-reported symptoms and severity, along with additional data such as sleep patterns or daily activities, personalized insights can be generated to assist in monitoring and diagnosing mental health disorders.

18. Sensor Fusion for Emotional State Detection: Cell phones equipped with multiple sensors, such as the accelerometer, GPS, microphone, and heart rate monitor, can collect data simultaneously. Sensor fusion techniques can combine data from these sensors to infer an individual's emotional state, which can then be compared to the DSM-5 criteria for relevant mental health disorders.

19. Social Media Analysis: Cell phones provide access to social media platforms, which individuals often use to express their thoughts, emotions, and experiences. Analyzing social media content, such as posts, comments, and interactions, can provide insights into an individual's mental health state. Natural language processing and sentiment analysis techniques can be applied to detect patterns indicative of mental health disorders.

20. Social Media Monitoring: Cell phones provide access to social media platforms where individuals often express their thoughts, feelings, and experiences. Analyzing social media posts using natural language processing and sentiment analysis techniques can provide insights into mental health symptoms and align them with DSM-5 criteria.

21. Speech and Language Analysis: Cell phones have the capability to record and analyze speech patterns and language use. By applying natural language processing (NLP) techniques, voice recordings can be analyzed to identify linguistic markers and speech patterns associated with specific mental health disorders, such as schizophrenia or depression.

22. Virtual Reality (VR) Assessment: Cell phones can support VR experiences, offering immersive environments for mental health assessment. VR scenarios can be designed to evoke specific emotional responses, allowing clinicians to observe and evaluate an individual's reactions to different stimuli, helping to assess symptoms related to anxiety, phobias, or post-traumatic stress disorder. Cell phones can be combined with VR technology to create immersive environments for mental health assessments. Virtual reality scenarios can be designed to elicit specific responses and behaviors related to DSM-5 criteria, providing clinicians with a controlled and standardized environment for diagnosis.

23. Voice Analysis for Emotion Detection: Cell phones can utilize voice analysis algorithms to detect emotional patterns and changes in speech characteristics. By analyzing factors like pitch, tone, speech rate, and word choice, it is possible to identify emotional states that align with DSM-5 criteria for specific mental health disorders.

Examples Relevant to Minnesota Multiphasic Personality Inventory (MMPI):

1. App-Based Psychological Assessments: Developing mobile applications that incorporate the MMPI criteria along with other validated psychological assessments can provide a more comprehensive and personalized diagnostic tool. These apps can collect data on mood, behavior, cognitive functioning, and other relevant factors, allowing for ongoing monitoring and assessment of mental health.

2. Behavioral Monitoring: The cell phone's accelerometer and GPS can be used to track an individual's movement patterns and activity levels throughout the day. By integrating this data with the MMPI responses, it becomes possible to identify correlations between specific behavioral patterns and mental health symptoms.

3. Biometric Data Integration: The cell phone's biometric sensors, such as heart rate monitors or skin conductance sensors, can be used to capture physiological responses during the administration of the MMPI. By correlating these biometric measurements with the individual's questionnaire responses, it may be possible to detect physiological markers associated with specific mental health disorders.

4. Contextual Data Collection: Cell phones can collect contextual data such as location, social interactions, and daily routines. By combining this data with MMPI responses, patterns can be identified that relate environmental or situational factors to specific mental health symptoms or triggers.

5. Ecological Momentary Assessment (EMA): Using the cell phone's features, individuals can be prompted at random intervals to answer specific questions related to their mental health symptoms and experiences. EMA can provide real-time data on fluctuations in symptoms, triggers, and contextual factors that impact mental health, complementing the information obtained from the MMPI.

6. Facial Expression Recognition: The cell phone's camera can be employed to capture facial expressions while an individual responds to the MMPI statements. Facial expression recognition software, based on machine learning and computer vision techniques, can analyze facial cues and provide insights into emotional states and potential mental health conditions.

7. Keyboard, Touchscreen, and Typing Patterns: Analyzing an individual's typing patterns, touchscreen usage, and keystrokes on the cell phone's keyboard can offer insights into their cognitive and emotional states. Advanced algorithms can detect variations in typing speed, accuracy, and patterns that may be indicative of mental health conditions.

8. Mobile App Administration: Develop a mobile application that administers the MMPI questionnaire in a user-friendly and interactive way. The app can guide individuals through the assessment process, provide real-time feedback, and collect data for analysis. This approach enables remote administration and monitoring of mental health symptoms.

9. Multimodal Data Fusion: Integrating data from multiple sensors on the cell phone, such as voice analysis, facial expression recognition, accelerometer data, and GPS information, can enable a comprehensive analysis of an individual's mental health status. Machine learning algorithms can be employed to fuse and analyze these multimodal data sources, providing a more holistic and accurate diagnostic assessment.

10. Sleep Monitoring: Utilizing the cell phone's sensors, such as the accelerometer and microphone, sleep patterns can be monitored and analyzed. Disruptions in sleep architecture or the presence of sleep-related symptoms can be correlated with the MMPI responses to provide a more comprehensive understanding of an individual's mental health status.

11. Social Media Analysis: Integrating social media data with the MMPI responses can provide valuable insights into an individual's mental health. Natural language processing and sentiment analysis techniques can be employed to analyze social media posts, comments, and interactions, helping identify patterns related to mental health symptoms or risk factors.

12. Voice Analysis: The cell phone's microphone can be utilized to capture and analyze an individual's voice patterns during the administration of the MMPI. Advanced voice analysis algorithms can be developed to detect subtle vocal characteristics associated with specific mental health disorders, providing additional diagnostic information.

Examples Relevant to DSM-5:

1. Ambient Environment Analysis: The cell phone's microphone and other environmental sensors are employed to analyze the user's ambient environment, including background noise levels, conversations, and general living conditions. By comparing this data to the environmental factors specified in the DSM-5, the application can identify potential triggers or stressors associated with mental health disorders. Users receive recommendations on modifying their environment for better mental well-being.

2. Automated Facial Expression Analysis: A mobile application utilizes the cell phone's camera to capture and analyze facial expressions of a user during video calls or recorded videos. The application compares the user's facial expressions to predefined patterns associated with different mental health disorders, as outlined in the DSM-5 criteria. The analysis provides feedback or alerts to the user and/or healthcare professionals regarding potential mental health conditions.

3. Behavior Monitoring and Pattern Recognition: A mobile application monitors the user's behavior patterns using various sensors on the cell phone, including the camera and microphone. The application utilizes machine learning algorithms to identify behavioral changes, such as social withdrawal, changes in speech patterns, or abnormal facial expressions. These changes are then compared to the DSM-5 criteria to detect potential mental health disorders and provide relevant feedback or alerts.

4. Biometric Data Analysis: A mobile application collects and analyzes biometric data from the cell phone's sensors, such as heart rate, skin conductivity, and sleep patterns. By correlating these physiological indicators with the diagnostic criteria outlined in the DSM-5, the application can detect patterns and anomalies associated with various mental health disorders. Users receive personalized feedback and recommendations based on their biometric data analysis.

5. Biometric Sensor Integration/Fusion: The cell phone incorporates additional biometric sensors, such as heart rate monitors or electrodermal activity sensors, to collect physiological data. By analyzing changes in these biometric parameters alongside behavioral patterns, the application can identify potential indicators of mental health disorders, such as heightened stress levels or autonomic nervous system dysregulation.

6. Cognitive Assessment and Games: A mobile application presents cognitive assessment tasks and games that are specifically designed to evaluate cognitive functions related to mental health disorders. Utilizing the cell phone's touchscreen, accelerometer, and other sensors, the application measures response times, accuracy, and cognitive performance. The collected data is then compared to the DSM-5 criteria to identify potential cognitive impairments associated with mental health disorders.

7. Contextual Data Analysis: A mobile application collects contextual data from the cell phone, such as GPS location, social media activity, phone usage patterns, and audio/video recordings. By analyzing this data alongside the DSM-5 criteria, the application can identify correlations between certain contextual factors and mental health disorders. This information can be used to provide personalized recommendations, early intervention, or professional referrals.

8. Data Fusion for Comprehensive Assessment: A mobile application integrates data from multiple sources, including the camera, microphone, GPS, sleep trackers, and activity monitors on the cell phone. By fusing and analyzing these diverse datasets, the application evaluates patterns, correlations, and deviations from normal behavior. The collected data is then compared with the DSM-5 criteria to identify potential mental health disorders, supporting early detection and intervention.

9. Environmental Context Analysis: A mobile application utilizes the cell phone's sensors, including GPS, ambient light, noise level, and activity trackers, to gather contextual information about the user's environment. By combining this data with DSM-5 criteria, the application assesses how environmental factors contribute to the development or exacerbation of mental health disorders. It provides personalized recommendations to modify the environment for better mental well-being.

10. Environmental Context Detection: The cell phone's sensors, such as ambient light sensors or barometers, are used to detect environmental factors that may impact mental health, such as light levels, noise levels, or air quality. By correlating these environmental data with the user's self-reported symptoms and behavioral patterns, the application can identify potential triggers or aggravators of mental health disorders. It offers personalized suggestions for creating healthier environments.

11. Facial Expression Analysis: The cell phone's camera captures the user's facial expressions during daily activities. Using computer vision and facial recognition algorithms, the application analyzes the user's emotional expressions and compares them to known patterns associated with mental health disorders. It provides real-time feedback on the user's emotional state and identifies potential indicators of specific disorders.

12. Facial Recognition and Emotion Analysis: A mobile application utilizes advanced facial recognition algorithms to analyze facial expressions captured by the cell phone's camera. By comparing the detected facial expressions with the emotional criteria outlined in the DSM-5, the application can identify potential indicators of mental health disorders. This real-time analysis provides immediate feedback and prompts the user to seek professional evaluation or treatment.

13. Location-Based Triggers: The cell phone's GPS and location tracking capabilities are utilized to identify locations or environments that trigger certain mental health symptoms. By correlating the user's location data with self-reported symptoms and the DSM-5 criteria, the application can identify patterns and provide personalized recommendations to avoid or manage triggering locations. Additionally, users can be tracked to see if they follow recommended behaviors, e.g. avoiding certain locations.

14. Longitudinal Data Tracking: The mobile application allows users to track and record their mental health symptoms, moods, and behaviors over time. Using self-reporting and user input, combined with data from the cell phone's sensors, the application creates a longitudinal profile of the user's mental health. By comparing this data to the DSM-5 criteria, the application can provide personalized insights, trends, and early warning signs of potential mental health disorders.

15. Machine Learning and Natural Language Processing: A mobile application employs machine learning algorithms and natural language processing techniques to analyze text messages, social media posts, and other written or verbal communications captured by the cell phone's microphone. By identifying patterns in language use, sentiment, and key phrases, the application detects potential indicators of mental health disorders specified in the DSM-5. It provides personalized feedback, resources, and recommendations for further evaluation or treatment.

16. Machine Learning-Based Risk Assessment: The application employs machine learning algorithms to analyze a wide range of data collected from the cell phone, including sensor data, user interactions, and self-reported symptoms. By training the algorithms on large datasets of individuals diagnosed with mental health disorders, the application can predict the risk of developing specific disorders based on individual patterns and provide early intervention or preventive measures.

17. Multimodal Data Fusion: The application integrates and analyzes data from multiple sources, such as biometric data, social media activity, voice recordings, and location information. By combining these different data modalities and applying machine learning algorithms, the application can generate comprehensive profiles of the user's mental health status. It provides holistic insights and predictions regarding potential mental health disorders, helping users and healthcare professionals make informed decisions.

18. Sensor-Based Behavioral Monitoring: The cell phone's sensors, such as the accelerometer, gyroscope, and GPS, are used to monitor the user's physical activity, mobility, and location patterns. By applying machine learning algorithms to this sensor data, the application can detect changes in behavior and activity levels that may indicate the presence of mental health disorders. Users receive notifications or alerts if significant deviations are detected.

19. Sleep Pattern Analysis: The cell phone's accelerometer and gyroscope can be utilized to monitor the user's sleep patterns, movement, and disturbances during the night. By analyzing sleep duration, quality, and disruptions, alongside self-reported sleep-related symptoms, the application can identify potential sleep disorders that often co-occur with mental health disorders. It provides recommendations for improving sleep hygiene and potential referrals for further evaluation.

20. Social Interaction Assessment: The mobile application uses the cell phone's communication features, such as call logs, text messages, and social media interactions, to assess the user's social interactions and communication patterns. By analyzing the content, frequency, and quality of these interactions, the application can identify deviations from normal behavior or social withdrawal, which may indicate potential mental health disorders.

21. Social Interaction Monitoring: A mobile application monitors the user's social interactions and communication patterns, including phone calls, text messages, and social media interactions. By analyzing the content, sentiment, and frequency of these interactions, the application identifies potential signs of mental health disorders as defined by the DSM-5. The user receives notifications or alerts if concerning patterns are detected, encouraging them to seek professional help.

22. Social Media and Text Analysis: A mobile application integrates with the user's social media accounts and messaging apps to analyze their digital footprint. By using natural language processing and sentiment analysis techniques, the application identifies keywords, linguistic patterns, and emotional expressions that align with the DSM-5 criteria for mental health disorders. It provides users with insights into their online behavior and potential risk factors.

23. Social Media Monitoring: The application integrates with the user's social media accounts to monitor their online activities, posts, and interactions. By analyzing content, sentiment, and engagement metrics, the application can detect signs of emotional distress, social isolation, or negative affect associated with mental health disorders. It provides insights and alerts based on these social media monitoring results.

24. Speech Pattern Analysis: The cell phone's microphone captures the user's speech patterns and linguistic cues during conversations or recorded audio. Advanced natural language processing algorithms analyze the user's speech for abnormalities, such as speech rate, word choice, or syntactic patterns that deviate from their baseline or established linguistic norms. These deviations can serve as potential markers for mental health disorders.

25. Virtual Reality Exposure Therapy: A mobile application combines the cell phone's camera, microphone, and virtual reality capabilities to simulate controlled environments for exposure therapy under supervision by a licensed mental health provider. The application presents virtual scenarios designed to provoke specific reactions or triggers related to mental health disorders, as outlined in the DSM-5. Through real-time monitoring of physiological responses, facial expressions, and audio cues, the application provides insights into the user's reactions and helps assess the presence and severity of relevant disorders.

26. Voice Analysis and Speech Patterns: A mobile application records and analyzes the user's speech patterns, tone of voice, and vocal characteristics using the cell phone's microphone. By applying voice analysis techniques and machine learning algorithms, the application detects deviations from normal speech patterns associated with mental health disorders specified in the DSM-5. The user receives personalized insights and recommendations based on the analysis.

27. Voice Analysis: The cell phone's microphone records the user's voice during phone calls, conversations, or even dedicated voice recordings. Advanced voice analysis algorithms are applied to detect changes in pitch, tone, speed, and other vocal characteristics that may correlate with symptoms of mental health disorders. The application provides insights and alerts based on these voice analysis results.

28. Voice Modulation and Speech Analysis: A mobile application uses the cell phone's microphone to capture and analyze the user's voice during phone calls or voice recordings. The application employs advanced algorithms to detect changes in speech patterns, tone, and voice modulation that may correlate with specific mental health disorders. The analysis is compared against the criteria outlined in the DSM-5, providing insights and potential diagnoses to the user and/or healthcare professionals.

Examples Relevant to International Classification of Diseases, Tenth Revision (ICD-10):

1. Activity and Behavior Monitoring: The sensors within a cell phone, such as accelerometers and GPS, can track and analyze an individual's activity levels, movement patterns, and behavioral changes. By correlating these data with ICD-10 criteria, it may be possible to identify and monitor symptoms of disorders like attention-deficit/hyperactivity disorder (ADHD) or obsessive-compulsive disorder (OCD).

2. Activity and Movement Tracking: The cell phone's accelerometer and GPS features can be leveraged to monitor an individual's activity and movement patterns throughout the day. Changes in activity levels, sleep patterns, or mobility may indicate symptoms of depression, mania, or other mood disorders.

3. Ambient Environment Monitoring: The cell phone's sensors can be utilized to monitor ambient environmental factors that may influence mental health. This can include tracking noise levels, light exposure, or air quality, which may contribute to conditions like anxiety, sleep disorders, or seasonal affective disorder (SAD).

4. Biometric Data Integration: Cell phones increasingly incorporate biometric sensors, such as heart rate monitors and electrodermal activity sensors. By collecting and analyzing these physiological signals alongside ICD-10 criteria, it may be possible to detect physiological markers associated with conditions like panic disorder, sleep disorders, or substance use disorders.

5. Biometric Measurements: The cell phone's built-in sensors, such as heart rate sensors or fingerprint scanners, can be used to collect biometric data. By analyzing physiological parameters, such as heart rate variability, stress levels, or skin conductance, it may be possible to infer mental health states or identify symptoms associated with anxiety disorders, post-traumatic stress disorder (PTSD), or attention-deficit/hyperactivity disorder (ADHD).

6. Biometric Mood Tracking: By leveraging the cell phone's sensors and data analysis capabilities, it is possible to develop algorithms that detect and track changes in an individual's mood. For example, by combining data from the accelerometer, GPS, and social media usage, it may be possible to create personalized mood profiles and detect patterns associated with mood disorders like depression or bipolar disorder.

7. Cognitive Assessments: Utilizing the cell phone's touchscreen and sensors, novel cognitive assessment tasks can be developed to evaluate various cognitive functions. These tasks may involve memory, attention, problem-solving, or executive functioning, providing insights into conditions like dementia, cognitive impairment, or attention-deficit/hyperactivity disorder (ADHD).

8. Cognitive Function Assessment: Cell phone applications can be developed to administer cognitive tasks and assessments, such as memory tests, attention tests, or problem-solving exercises. By analyzing performance data collected through these applications, it may be possible to identify cognitive impairments associated with conditions like dementia, traumatic brain injury, or attention-deficit/hyperactivity disorder (ADHD).

9. Contextual Data Integration: The cell phone can collect contextual data from various sources, such as location information, calendar events, or communication patterns. By integrating this data with the ICD-10 criteria, it may be possible to identify correlations between specific contexts and mental health symptoms. For example, detecting associations between certain locations, time of day, or social interactions with conditions like phobias, obsessive-compulsive disorder (OCD), or postpartum depression.

10. Environmental Monitoring: The cell phone's sensors, including the microphone, GPS, and accelerometer, can be used to gather data on an individual's environmental context. By analyzing factors such as ambient noise levels, movement patterns, and location data, it may be possible to correlate environmental triggers with mental health symptoms or episodes, aiding in the diagnosis of conditions like post-traumatic stress disorder (PTSD) or phobias.

11. Environmental Triggers and Contextual Analysis: Cell phones can collect various contextual information, including location, time, and environmental factors through sensors and data connections. By integrating this contextual data with ICD-10 criteria, it may be possible to identify specific triggers or situational factors that contribute to the onset or exacerbation of certain mental health disorders, such as post-traumatic stress disorder (PTSD) or specific phobias.

12. Facial Expression Analysis: The cell phone's camera can be used to capture and analyze facial expressions of individuals. Advanced computer vision algorithms can be employed to detect and analyze emotional cues, such as changes in facial expressions associated with specific mental health disorders. This data, combined with ICD-10 diagnostic criteria, can aid in the assessment and diagnosis of disorders like depression, anxiety, or schizophrenia.

13. Facial Expression Analysis: The cell phone's camera can be used to capture facial expressions and analyze them using computer vision algorithms. By examining facial expressions for signs of emotional distress, such as sadness, fear, or anger, it may be possible to assess conditions like depression, anxiety disorders, or bipolar disorder.

14. Physiological Monitoring: The cell phone can be paired with external wearable devices, such as heart rate monitors or electrodermal activity sensors, to collect physiological data. By analyzing changes in physiological parameters during different activities or situations, it may be possible to identify patterns indicative of mental health disorders, such as panic disorder, generalized anxiety disorder, or obsessive-compulsive disorder (OCD).

15. Sleep Monitoring and Analysis: Many cell phones have built-in sensors, such as accelerometers, that can be utilized to track movement and sleep patterns. By combining this data with audio recordings of sleep sounds and sleep quality ratings reported by users, it may be possible to assess sleep disorders, such as insomnia or sleep apnea, which are often associated with mental health conditions.

16. Social Interaction Analysis: Cell phones are often used for communication and social interactions. Analyzing text messages, phone call patterns, and social media activity can provide insights into an individual's social functioning and relationships. By comparing these data with ICD-10 criteria, it may be possible to identify social impairment associated with conditions such as autism spectrum disorder or social anxiety disorder. The cell phone's microphone can be used to capture audio during social interactions, while natural language processing algorithms analyze the content. By examining communication patterns, social cues, and language use, it may be possible to identify difficulties in social interactions associated with conditions like autism spectrum disorder (ASD), social anxiety disorder, or schizophrenia.

17. Social Interaction Monitoring: The cell phone's communication features, including calls, text messages, and social media interactions, can be analyzed to assess an individual's social interactions and communication patterns. Changes in communication frequency, content, or social network dynamics may provide indicators of mental health conditions such as social anxiety, borderline personality disorder, or autism spectrum disorders.

18. Social Media Analysis: By integrating with social media platforms, the cell phone can analyze an individual's online behavior, posts, and interactions. Machine learning algorithms can identify linguistic cues, sentiment patterns, or social network characteristics that may correlate with mental health conditions such as social anxiety, depression, or eating disorders. Cell phones provide access to social media platforms where individuals express their thoughts, emotions, and behaviors. By analyzing social media content, including text, images, and videos, using natural language processing and sentiment analysis techniques, it may be possible to identify signs of mental health disorders such as depression, anxiety, or eating disorders.

19. Speech and Language Analysis: The cell phone's microphone can be used to capture speech samples, which can then be analyzed using natural language processing and machine learning algorithms. By examining speech patterns, semantic content, and linguistic markers, it may be possible to detect abnormalities associated with conditions like schizophrenia, aphasia, or cognitive impairment.

20. Speech Pattern Analysis: The cell phone's microphone can be utilized to analyze speech patterns and detect linguistic markers associated with specific mental health disorders. By applying natural language processing and machine learning techniques, it may be possible to identify speech characteristics related to conditions such as schizophrenia, cognitive impairments, or mood disorders.

21. Visual and Audio Stimulus Response: The cell phone's camera and microphone can be used to present visual and audio stimuli and record an individual's physiological responses, such as changes in pupil dilation or heart rate variability. By analyzing these responses alongside ICD-10 criteria, it may be possible to assess emotional reactivity and sensory processing abnormalities related to conditions like post-traumatic stress disorder (PTSD) or autism spectrum disorder (ASD).

22. Vocal Analysis: The cell phone's microphone can be utilized to capture and analyze changes in vocal characteristics, including tone, pitch, and speech patterns. Machine learning algorithms can be applied to identify patterns associated with mental health disorders, such as changes in speech indicative of bipolar disorder or thought disorder. This approach could provide additional diagnostic information based on ICD-10 criteria. In addition to speech pattern analysis, the cell phone's microphone can be used to analyze various vocal characteristics such as tone, pitch, and speed. By employing machine learning algorithms, voice analysis can provide insights into mental health conditions like schizophrenia, mood disorders, or substance abuse. The cell phone's microphone can be used to record and analyze an individual's voice for markers of mental health disorders. By examining features such as pitch, tone, speech rate, and vocal intensity, it may be possible to detect symptoms associated with conditions like depression, schizophrenia, or Parkinson's disease. Facial Expression Analysis: The cell phone's camera can be used to capture and analyze facial expressions in real-time. Advanced facial recognition algorithms combined with emotion detection techniques can help identify patterns and markers associated with mental health disorders such as depression, anxiety, or bipolar disorder.

Examples Relevant to Research Domain Criteria (RDoC):

1. Behavioral Monitoring and Intervention: The cell phone can be used as a platform for continuous behavioral monitoring and interventions. Machine learning algorithms can analyze various sensor data, such as movement patterns, social interactions, and communication patterns, to detect deviations from baseline behavior and provide timely interventions or alerts when indicators of mental health disorders are detected.

2. Cognitive Assessment: Utilizing the cell phone's touchscreen and sensors, it becomes possible to administer cognitive tasks and assessments remotely. Mobile applications can be developed to assess attention, memory, executive function, and other cognitive domains, providing objective measures of cognitive functioning relevant to mental health disorders.

3. Contextual Assessments: The cell phone's GPS and other sensors can provide contextual information about an individual's location, activities, and environmental factors. By integrating this data with RDoC criteria, it becomes possible to assess how different contexts and settings influence mental health symptoms and functional impairments.

4. Contextual Monitoring: The GPS capabilities of a cell phone can track an individual's location and movement patterns. By linking this information with other sensor data, such as the accelerometer or ambient noise levels, it becomes feasible to examine how environmental contexts impact mental health. For example, assessing the relationship between specific locations, social interactions, and emotional states can contribute to understanding conditions related to fear and anxiety.

5. Ecological Momentary Assessment (EMA): Using the cell phone as a platform for EMA, individuals can provide real-time self-reports about their experiences, emotions, and behaviors in their natural environment. These reports can be supplemented with objective data captured by the phone's sensors, such as voice recordings or images, to provide a comprehensive picture of an individual's mental state across different RDoC domains.

6. Ecological Momentary Assessment: The cell phone can be used to implement ecological momentary assessment techniques, where individuals are prompted to provide real-time assessments of their mental state, activities, and environmental factors throughout the day. This approach allows for capturing momentary fluctuations in mental health and provides a more accurate representation of an individual's experiences.

7. Environmental Monitoring: The cell phone's sensors and connectivity can be used to monitor environmental factors that may impact mental health, such as noise levels, ambient light, or air quality. By considering the influence of the environment on mental health, a more comprehensive assessment can be achieved using the RDoC framework.

8. Environmental Triggers: The cell phone's sensors, including the microphone and GPS, can help identify environmental triggers that may contribute to mental health symptoms. By collecting data on ambient noise levels, location, and other contextual factors, correlations can be explored between environmental factors and specific dimensions of mental health.

9. Facial Expression Analysis: The cell phone's camera can be utilized to capture facial expressions and analyze them using computer vision techniques. Advanced algorithms can detect and analyze facial expressions associated with various emotional states, providing valuable insights into emotional processes relevant to mental health disorders.

10. Longitudinal Data Collection: With the continuous use of cell phones in individuals' daily lives, it becomes possible to collect longitudinal data on behavior, communication patterns, environmental factors, and physiological responses. Long-term data collection can facilitate the identification of temporal patterns, predictive markers, and dynamic relationships between mental health and various RDoC domains.

11. Longitudinal Tracking: The cell phone's continuous data collection capabilities enable longitudinal tracking of mental health indicators over time. Long-term monitoring and analysis of sensor data, combined with RDoC criteria, can facilitate the identification of patterns, trajectories, and changes in mental health states.

12. Machine Learning and Predictive Models: By applying machine learning algorithms to the data collected from cell phone sensors, it becomes possible to develop predictive models for mental health outcomes. These models can identify patterns, risk factors, and early warning signs of mental health disorders based on sensor data and RDoC criteria.

13. Mood Tracking and Self-Reporting: Mobile applications can be developed to facilitate mood tracking and self-reporting of mental health symptoms. Through user-friendly interfaces, individuals can regularly report their mood, emotions, and other subjective experiences, which can be correlated with RDoC domains and used for assessment purposes.

14. Multimodal Behavioral Assessment: By integrating multiple features of a cell phone, including the camera, microphone, accelerometer, and GPS, it becomes possible to collect rich and diverse behavioral data. This data can be analyzed using machine learning algorithms to identify patterns and markers associated with different RDoC domains, such as cognition, emotion, social processes, or sensorimotor function.

15. Multimodal Data Fusion: Integrating data from multiple sensors and modalities on the cell phone, such as audio, visual, and physiological data, allows for multimodal data fusion. By combining different data sources, it becomes possible to uncover complex relationships and interactions between mental health indicators and RDoC domains.

16. Personalized Interventions: Leveraging the capabilities of a cell phone, personalized interventions can be delivered to individuals based on their specific mental health needs and RDoC profiles. These interventions can include psychoeducation, cognitive behavioral therapy modules, mindfulness exercises, or other therapeutic techniques accessible through mobile applications.

17. Physiological Monitoring: The cell phone's sensors, such as the accelerometer or heart rate monitor, can be used to collect physiological data in real-time. These measures can be correlated with other behavioral and self-reported data to investigate associations between physiological responses and RDoC domains, such as arousal, attention, or reward processing.

18. Physiological Response Assessment: The cell phone's sensors, such as the camera or heart rate monitor, can be used to capture physiological responses, such as changes in heart rate, skin conductance, or pupil dilation. These physiological markers can be linked to specific RDoC domains and provide insights into physiological processes underlying mental health disorders.

19. Sensor Fusion: By combining data from multiple sensors on the cell phone, such as the accelerometer, microphone, and GPS, it becomes possible to perform sensor fusion analysis. This approach allows for a comprehensive understanding of an individual's behavior, movement patterns, environmental context, and physiological responses, providing a holistic view of mental health indicators.

20. Sleep Monitoring: The cell phone's accelerometer and microphone can be used to monitor sleep patterns and disturbances. By analyzing movement patterns, ambient noise, and other physiological signals during sleep, it becomes possible to assess sleep quality, identify sleep disorders, and examine the relationship between sleep and mental health. Sleep disturbances are often associated with various mental health disorders, and analyzing sleep data in conjunction with RDoC domains can contribute to the assessment and understanding of these disorders.

21. Social Interaction Analysis: Leveraging the cell phone's communication features, such as call logs, messaging apps, and social media platforms, it becomes possible to analyze social interactions and communication patterns. Natural language processing and social network analysis techniques can be applied to understand social behavior, social relationships, and their association with mental health outcomes. This analysis can aid in understanding social deficits associated with certain mental health disorders and RDoC constructs related to social functioning.

22. Social Network Analysis: Leveraging the cell phone's communication features and social media data, social network analysis techniques can be employed to examine the structure and dynamics of an individual's social connections. This can provide insights into social processes related to mental health, such as social support networks, social influence, or social isolation. This analysis can help understand the influence of social relationships, social support, and social context on mental health outcomes.

23. Speech Analysis: The cell phone's microphone can be utilized to analyze speech patterns and characteristics. Advanced natural language processing algorithms can detect linguistic features, speech tempo, prosody, and other acoustic parameters that may be indicative of mental health disorders or specific RDoC domains.

24. Speech and Language Analysis: The cell phone's microphone can be utilized to capture and analyze speech patterns, including speech rate, tone, and linguistic characteristics. Natural language processing techniques can be applied to detect linguistic markers associated with specific RDoC domains, such as disruptions in cognition, social communication, or emotional expression.

25. Voice Analysis: In addition to speech analysis, the cell phone's microphone can be used to analyze voice characteristics such as pitch, tone, and quality. Voice analysis techniques can help identify vocal patterns associated with specific mental health conditions or RDoC constructs related to vocal communication and emotional expression.

Examples Relevant to Structured Clinical Interview for DSM Disorders (SCID)

1. Behavioral Pattern Analysis: By analyzing the data captured by the cell phone's accelerometer and GPS, it becomes possible to detect and quantify changes in physical activity, mobility patterns, and daily routines. Deviations from established behavioral patterns can serve as indicators of mental health disorders, aligning with RDoC constructs related to motor systems and circadian rhythms.

2. Cognitive Assessment: The cell phone can be used as a platform for administering cognitive tasks and assessments. Through the screen and input functionalities, various cognitive tests can be delivered, measuring attention, memory, executive functions, and other cognitive processes relevant to mental health disorders and RDoC constructs focusing on cognition.

3. Cognitive Bias Assessment: Utilizing the cell phone's screen, cognitive tasks can be designed to measure cognitive biases associated with mental health disorders. By presenting stimuli and recording responses, it becomes possible to evaluate attentional biases, interpretation biases, and other cognitive processes relevant to RDoC constructs related to cognitive systems.

4. Cognitive Performance Assessment: Utilizing the cell phone's touchscreen and processing capabilities, cognitive tasks and assessments can be administered to measure cognitive performance. These tasks can target specific cognitive domains related to mental health disorders, such as attention, memory, and executive functions, aligning with RDoC constructs related to cognitive systems.

5. Contextual Analysis: Leveraging the GPS capabilities of a cell phone, the RDoC criteria can be applied in conjunction with location data to understand how contextual factors, such as specific environments or situations, influence mental health. By analyzing location patterns and their correlation with mental health symptoms, it becomes possible to gain insights into the impact of context on various RDoC constructs.

6. Contextual Mood Monitoring: By combining data from the cell phone's sensors and self-reported mood assessments, it becomes possible to monitor individuals' mood fluctuations in different contexts. This contextual mood monitoring can shed light on the impact of environmental factors on emotional well-being, aligning with RDoC constructs related to emotion and environmental systems.

7. Data Integration and Machine Learning: By integrating data from multiple sensors on the cell phone, such as audio, visual, movement, and environmental data, one can apply machine learning algorithms to identify patterns, predictors, and early warning signs of mental health disorders based on RDoC constructs. This data integration approach enables a holistic and personalized assessment of mental health.

8. Ecological Momentary Assessment (EMA): Through the cell phone, ecological momentary assessment techniques can be employed to collect real-time data on individuals' thoughts, emotions, behaviors, and experiences in their natural environments. EMA provides a rich and dynamic picture of mental health symptoms and their relationship to RDoC constructs in real-world contexts. Through the use of mobile applications, individuals can be prompted to complete self-report assessments of their current mental state and experiences in real-time. This ecological momentary assessment allows for the capture of moment-to-moment fluctuations in mental health symptoms, providing valuable data aligned with RDoC constructs.

9. Emotion Recognition: Leveraging the camera on the cell phone, computer vision techniques can be employed to analyze facial expressions and detect emotional states. By using machine learning algorithms, the cell phone can recognize and quantify emotional expressions associated with mental health disorders and relevant RDoC constructs related to emotion and social systems. Using the cell phone's camera, facial expression recognition algorithms can be applied to detect and analyze emotional states. This allows for the assessment of emotional dysregulation and emotion-related RDoC constructs using visual cues captured through the device.

10. Environmental Contextualization: In addition to GPS data, the cell phone can gather information about the user's environmental context, such as weather conditions, noise levels, or social proximity. By integrating this contextual information with mental health assessments based on RDoC constructs, it becomes possible to explore how the environment influences symptom expression and disease progression.

11. Environmental Exposure Assessment: The cell phone's sensors and connectivity can be employed to monitor environmental exposures that may impact mental health. For example, the device can detect and record noise levels, air quality, or light exposure. By integrating environmental data with RDoC criteria, one can explore the relationship between environmental factors and mental health outcomes.

12. Environmental Triggers Detection: By integrating data from the cell phone's sensors and external data sources, such as weather APIs and air quality sensors, it becomes possible to identify environmental triggers that may exacerbate symptoms of mental health disorders. This information can be used to develop personalized interventions and strategies aligned with RDoC constructs related to stress and environmental systems.

13. Heart Rate Variability (HRV) Monitoring: The cell phone's built-in heart rate sensor can be used to measure heart rate variability, which is an indicator of autonomic nervous system activity and emotional regulation. By analyzing HRV data, it becomes possible to assess emotional dysregulation and its relationship to mental health disorders aligned with RDoC constructs focusing on emotion and arousal systems.

14. Linguistic Analysis: By analyzing text messages, emails, or other textual data stored on the cell phone, natural language processing techniques can be used to extract linguistic features associated with mental health disorders. These features include word usage, semantic content, and linguistic style, providing insights into cognitive and linguistic processes aligned with RDoC constructs.

15. Longitudinal Tracking and Ecological Validity: The cell phone's continuous data collection capabilities enable long-term tracking of mental health symptoms and behaviors in real-world settings. This longitudinal approach provides ecological validity and a comprehensive understanding of individuals' mental health trajectories aligned with RDoC constructs.

16. Machine Learning-based Symptom Prediction: By applying machine learning algorithms to longitudinal data collected through the cell phone, it becomes possible to predict the occurrence or severity of mental health symptoms aligned with RDoC constructs. This predictive approach can assist in early detection and intervention, facilitating personalized mental health care.

17. Movement and Activity Monitoring: Utilizing the cell phone's accelerometer, movement and activity patterns can be monitored. Changes in physical activity levels and movement patterns can provide valuable information about mood, energy levels, and motor abnormalities associated with mental health disorders and RDoC constructs related to motor systems.

18. Sensor Fusion: By combining data from multiple sensors on the cell phone, such as the accelerometer, microphone, and camera, it becomes possible to perform sensor fusion analysis. This integrated analysis can provide a comprehensive assessment of various RDoC constructs, capturing multimodal information and its relationship to mental health disorders.

19. Sleep Analysis: The cell phone's sensors, such as the accelerometer and ambient light sensor, can be utilized to track sleep patterns and quality. By analyzing sleep duration, sleep efficiency, and other sleep-related metrics, it becomes possible to assess sleep disturbances and their impact on mental health, aligning with RDoC constructs related to sleep-wakefulness regulation.

20. Social Interaction Assessment: By leveraging the cell phone's microphone and camera, it becomes possible to capture and analyze social interactions. Speech patterns, facial expressions, and social cues can be analyzed to evaluate social engagement, social cognition, and interpersonal processes associated with mental health disorders and relevant RDoC constructs.

21. Social Media Text Mining: Leveraging the cell phone's connectivity, social media text mining techniques can be applied to analyze text-based data from platforms like Twitter or Facebook. Natural language processing algorithms can extract and analyze mental health-related content, providing insights into individuals' experiences, thoughts, and emotions aligned with RDoC constructs.

22. Social Network Analysis: By accessing social media data through the cell phone, one can analyze social network patterns and interactions. This allows for the exploration of social support systems, social connectivity, and social behavior as they relate to mental health disorders and RDoC constructs focusing on social processes. By analyzing social network data from the cell phone, such as contact lists, call logs, and text message metadata, it becomes possible to map social relationships and identify social support networks. This analysis can provide insights into social functioning and social relationship patterns associated with mental health disorders aligned with RDoC constructs related to social processes.

23. Speech Analysis: The cell phone's microphone can be utilized to analyze speech patterns and characteristics, including pitch, tone, speech rate, and language use. By applying natural language processing and machine learning algorithms, it is possible to identify linguistic markers associated with specific mental health disorders or RDoC constructs related to language and communication.

24. Speech Prosody Analysis: Apart from linguistic content, the cell phone's microphone can capture speech prosody, including tone, rhythm, and intonation. By analyzing these acoustic features using machine learning techniques, it becomes possible to identify patterns associated with mental health disorders and RDoC constructs related to communication systems.

25. Stress Monitoring: The cell phone's sensors, such as the accelerometer and heart rate monitor, can be utilized to measure physiological markers of stress. By analyzing changes in heart rate, activity levels, and other stress-related indicators, one can assess stress reactivity and regulation within the context of RDoC constructs.

26. Virtual Reality-based Assessments: By leveraging the cell phone's processing power and display capabilities, virtual reality environments can be created to simulate real-world situations that elicit specific responses associated with mental health disorders. These immersive assessments can provide ecologically valid data aligned with RDoC constructs related to various domains of functioning.

27. Voice and Speech Analysis: In addition to speech prosody, the cell phone's microphone can be used for voice and speech analysis. Advanced algorithms can be employed to detect changes in speech patterns, vocal characteristics, and language use, providing insights into mental health disorders aligned with RDoC constructs related to communication and language systems.

Examples Relevant to Structured Clinical Interview for DSM Disorders (SCID)

1. Audio Recording for Interview Analysis: The cell phone's microphone can be used to record SCID interviews conducted by mental health professionals. These audio recordings can later be analyzed for more accurate and consistent evaluation, allowing for a comprehensive review of the interview data.

2. Behavioral Pattern Recognition: The cell phone's sensors can capture behavioral data, such as movement patterns, daily routines, or changes in physical activity levels. Machine learning algorithms can analyze these patterns and identify deviations that may be indicative of mental health disorders such as bipolar disorder or major depressive disorder.

3. Biometric Data Integration: The cell phone's biometric sensors, such as heart rate monitors or electrodermal activity sensors, can capture physiological data during the SCID interview. Integrating these biometric measures with SCID assessments can provide additional objective indicators of arousal, stress, or emotional responses related to mental health disorders.

4. Cognitive Function Assessment: Utilizing the cell phone's touchscreen, cognitive tasks and assessments can be administered remotely. These tasks can measure attention, memory, executive function, and other cognitive domains, helping in the assessment of mental health disorders such as attention-deficit/hyperactivity disorder (ADHD), dementia, or cognitive impairments.

5. Communication Analysis: By analyzing call logs, text message metadata, and email communication on the cell phone, linguistic patterns and communication styles can be assessed. This analysis can provide additional insights into individuals' speech and language use, potentially aligning with SCID criteria related to communication disorders.

6. Contextual Audio Analysis: The cell phone's microphone can be used to capture and analyze audio data during real-life situations, such as social interactions, public speaking, or exposure to triggers. Audio analysis techniques can provide insights into social anxiety, specific phobias, or auditory hallucinations associated with conditions like schizophrenia.

7. Contextual Data Integration: The cell phone's accelerometer, GPS, and other sensors can provide contextual data about an individual's daily activities, movement patterns, and environmental factors. By integrating this contextual information with SCID assessments, clinicians can gain a more comprehensive understanding of how external factors might influence an individual's mental health symptoms.

8. Digital Phenotyping: By collecting and analyzing various data streams from the cell phone, including app usage, typing patterns, social media activity, and location data, it becomes possible to develop digital phenotypes associated with mental health disorders. These phenotypes can supplement the SCID criteria and provide additional information for diagnostic purposes.

9. Ecological Momentary Assessment (EMA): Mobile applications can be developed to administer brief self-report assessments to individuals throughout their daily lives. These assessments can be designed to collect data related to symptoms, mood fluctuations, or other factors relevant to the SCID criteria. EMA data can provide valuable real-time information for clinicians to consider during the diagnostic process. Using the cell phone's notification system, individuals can be prompted to provide real-time assessments of their mood, emotions, or symptoms throughout the day. This data, combined with the SCID criteria, allows for a more dynamic and contextually rich evaluation of mental health disorders.

10. Ecosystem Monitoring: The cell phone can act as a hub to connect and integrate with other wearable devices or smart home technologies. This ecosystem can monitor various physiological and behavioral parameters, such as heart rate, sleep quality, physical activity, and medication adherence. By combining this data with SCID assessments, a more comprehensive understanding of an individual's mental health status can be achieved.

11. Environmental Contextualization: The cell phone's GPS, ambient light sensor, and other environmental sensors can provide contextual information about an individual's surroundings during SCID assessments. This data can help identify potential triggers or environmental factors that contribute to mental health symptoms, such as agoraphobia, specific phobias, or seasonal affective disorder (SAD).

12. Environmental Triggers Detection: The cell phone's GPS and environmental sensors can be employed to detect environmental triggers that may influence mental health symptoms. For example, by analyzing location data and environmental factors like air quality or noise levels, the system can identify correlations between certain environments and the exacerbation of symptoms related to anxiety or post-traumatic stress disorder (PTSD).

13. Facial Expression Analysis: The cell phone's camera can be used to capture facial expressions during the SCID interview. Facial expression analysis algorithms can then be employed to detect and analyze facial cues associated with various mental health disorders, such as depression, anxiety, or post-traumatic stress disorder (PTSD).

14. GPS and Location Tracking: The GPS feature on a cell phone can track individuals' movement patterns and geographical locations. This information can be integrated with SCID assessments to gather data on environmental factors that may influence mental health symptoms, such as agoraphobia or social anxiety disorder.

15. Heart Rate Variability Analysis: The cell phone's built-in heart rate sensor or compatible wearable devices can capture heart rate variability data, which reflects the autonomic nervous system's functioning. Analyzing these patterns can provide insights into stress levels, emotional regulation, and potential indicators of anxiety disorders, post-traumatic stress disorder (PTSD), or depression.

16. Machine Learning-Based Decision Support: By leveraging machine learning algorithms, data collected from cell phone features, such as audio recordings, video recordings, and sensor data, can be used to develop predictive models. These models can assist clinicians in diagnosing mental health disorders by analyzing patterns, identifying risk factors, and providing decision support based on aggregated data from multiple sources.

17. Movement and Gesture Analysis: Leveraging the cell phone's accelerometer and gyroscope, algorithms can analyze movement patterns and gestures during SCID assessments. This approach can help assess motor abnormalities associated with mental health disorders such as Parkinson's disease, catatonia, or movement-related side effects of medication.

18. Multimedia Data Analysis: The cell phone's camera, microphone, and other multimedia capabilities can capture visual and auditory cues that may be relevant to mental health disorders. For example, facial expression analysis, voice sentiment analysis, or image recognition algorithms can assist in identifying signs of depression, anxiety, or other mood disorders.

19. Sensor Data for Behavioral Monitoring: The cell phone's accelerometer and other built-in sensors can capture data on movement, physical activity, sleep patterns, and other behavioral indicators. These data can be used to monitor changes in behavior that may be relevant to specific SCID criteria, such as changes in sleep patterns for diagnosing mood disorders.

20. Sleep Pattern Analysis: The cell phone's accelerometer and gyroscope can be used to monitor and analyze an individual's sleep patterns. By tracking movement and positioning during sleep, as well as ambient noise levels using the microphone, algorithms can provide insights into sleep quality and disturbances that may be associated with certain mental health disorders like insomnia or sleep-related disorders.

21. Social Interaction Analysis: Mobile apps or software can be developed to analyze text messages, social media interactions, and phone call logs captured on the cell phone. Natural language processing and social network analysis techniques can help identify patterns of social interaction and communication styles that align with specific mental health disorders, such as social anxiety disorder or personality disorders.

22. Social Media Analysis: By accessing social media data through the cell phone, algorithms can analyze posts, comments, and other digital interactions to identify linguistic, emotional, or behavioral patterns that align with specific mental health disorders. This approach can provide additional information to support the diagnostic process and track symptom progression over time.

23. Social Network Analysis: By analyzing data from the cell phone's contact list, call logs, and social media apps, algorithms can perform social network analysis to identify patterns of social support, social isolation, or dysfunctional social interactions. These patterns can provide valuable information for assessing mental health disorders such as social anxiety disorder, major depressive disorder, or borderline personality disorder.

24. Speech Analysis: The cell phone's microphone can be used to analyze speech patterns and detect linguistic markers associated with various mental health disorders. Natural language processing and machine learning techniques can be applied to assess factors such as speech rate, fluency, intonation, and content, providing insights into conditions like schizophrenia, bipolar disorder, or cognitive disorders.

25. Speech and Voice Analysis: The cell phone's microphone can be utilized to analyze speech patterns, voice characteristics, and linguistic markers during the SCID interview. Advanced algorithms and machine learning techniques can be applied to identify speech features associated with specific mental health disorders, such as patterns indicative of schizophrenia or bipolar disorder.

26. Speech Emotion Recognition: Utilizing the cell phone's microphone, advanced algorithms can be employed to recognize and analyze emotional states expressed through speech during the SCID interview. This approach can provide additional insights into the emotional experiences of individuals, aiding in the assessment of mood disorders and other emotional disturbances.

27. Video Recording for Behavioral Observations: The cell phone's camera can capture video recordings during the SCID interview process. This can enable clinicians to observe non-verbal cues, facial expressions, body language, and other behavioral indicators relevant to the diagnostic process.

Examples Relevant to Mini International Neuropsychiatric Interview (MINI)

1. Activity and Movement Tracking: The cell phone's accelerometer and GPS capabilities can track an individual's physical activity, movement patterns, and location. By analyzing activity levels, sleep patterns, and mobility, algorithms can identify disruptions or anomalies that may be associated with mental health disorders like insomnia, schizophrenia, or agitation.

2. Biometric Markers: The cell phone's sensors, such as the camera or fingerprint scanner, can be used to measure biometric markers associated with mental health disorders. For instance, changes in facial temperature, heart rate variability, or skin conductance captured by the cell phone's sensors can provide insights into stress, anxiety, or emotional dysregulation.

3. Biometric Measurements: Cell phones equipped with additional sensors, such as heart rate monitors or electrodermal activity sensors, can collect physiological data during the MINI assessment. By analyzing changes in heart rate variability, skin conductance, or other biometric measures, algorithms can identify physiological markers associated with mental health disorders like panic disorder, PTSD, or substance abuse disorders.

4. Contextual Mood Assessment: Through the cell phone's various sensors and user input, algorithms can assess an individual's mood in real-time during the MINI interview. This can be done by analyzing factors such as physical activity, voice modulation, social interactions, and self-reported mood ratings. This approach can provide additional information for diagnosing mood disorders like major depressive disorder or bipolar disorder.

5. Contextualized Self-Reporting: Through the cell phone's user interface, individuals can provide self-reports related to their mental health symptoms and experiences. Novel mobile apps can integrate the MINI assessment questions within a user-friendly interface, allowing individuals to provide real-time contextualized self-reports, such as mood ratings, stress levels, or symptom severity. This approach enhances the accuracy and ecological validity of self-reported data.

6. Daily Activity and Behavior Monitoring: The cell phone's various sensors can track an individual's daily activities, including phone usage patterns, app usage, mobility, and social interactions. By leveraging machine learning algorithms, patterns and changes in behavior can be analyzed to detect abnormalities or deviations associated with mental health disorders, such as obsessive-compulsive disorder (OCD), addiction disorders, or mood disorders.

7. Environmental Contextualization: The cell phone's sensors, including GPS and ambient light sensors, can provide contextual information about an individual's environment during the MINI assessment. By considering factors such as location, time of day, weather conditions, or ambient light levels, algorithms can assess how environmental factors may influence mental health symptoms or disorders, such as seasonal affective disorder (SAD), agoraphobia, or environmental sensitivity.

8. Environmental Noise Monitoring: The cell phone's microphone can also be used to monitor environmental noise levels during the MINI assessment. Excessive noise exposure or sensitivity to noise can be associated with mental health disorders like anxiety disorders, attention-deficit/hyperactivity disorder (ADHD), or post-traumatic stress disorder (PTSD). The cell phone's microphone can be used to monitor the surrounding environmental noise levels during the MINI interview. By analyzing noise patterns and decibel levels, algorithms can identify potential correlations between noise exposure and mental health symptoms or disorders such as attention-deficit/hyperactivity disorder (ADHD), anxiety disorders, or stress-related conditions.

9. Facial Expression Analysis: The cell phone's camera can be used to capture facial expressions during the MINI assessment. Advanced computer vision algorithms can then analyze these facial expressions to detect emotional states, such as sadness, anger, or anxiety. This information can provide additional insights into mood disorders and other related conditions. The cell phone's camera can be utilized to capture facial expressions during the MINI assessment. Advanced facial recognition algorithms can analyze facial cues, such as micro expressions, emotional intensity, or changes in facial muscle activity, to detect indicators of mental health disorders, including depression, bipolar disorder, or post-traumatic stress disorder (PTSD).

10. Location-Based Contextualization: The cell phone's GPS capabilities can provide information about an individual's location during the MINI assessment. This contextual data can be used to identify potential environmental factors that may contribute to mental health symptoms or trigger certain disorders, such as agoraphobia, panic disorder, or post-traumatic stress disorder (PTSD).

11. Location-Based Triggers: By utilizing the cell phone's GPS capabilities, algorithms can identify location-based triggers for mental health symptoms. For example, specific locations or contexts may trigger anxiety or panic attacks in individuals with phobias, agoraphobia, or post-traumatic stress disorder (PTSD).

12. Movement and Gesture Analysis: The cell phone's accelerometer and gyroscope can capture movement and gesture data during the MINI assessment. Advanced motion tracking algorithms can analyze motor abnormalities, repetitive movements, or unusual gestures that may be linked to mental health disorders like Tourette's syndrome, obsessive-compulsive disorder (OCD), or motor tics.

13. Physical Activity Monitoring: The cell phone's accelerometer and GPS capabilities can track an individual's physical activity and movement patterns. By analyzing these data, algorithms can identify changes in activity levels or deviations from normal routines, which may indicate symptoms of mental health disorders such as depression, anxiety, or obsessive-compulsive disorder (OCD).

14. Sleep Monitoring: The cell phone's accelerometer and microphone can be utilized to monitor sleep patterns and behaviors. Algorithms can analyze data on sleep duration, sleep quality, snoring, and other related factors to help assess sleep disorders, such as insomnia, sleep apnea, or parasomnias.

15. Social Interaction Analysis: By leveraging the cell phone's microphone and camera, algorithms can analyze an individual's social interactions during the MINI assessment. This analysis can include aspects such as speech patterns, social cues, and non-verbal communication, providing insights into conditions like autism spectrum disorder, social anxiety, or personality disorders.

16. Social Interaction Monitoring: By leveraging the cell phone's microphone and communication apps, algorithms can analyze social interactions and communication patterns. This can provide insights into the quality of interpersonal relationships, social withdrawal, or communication difficulties associated with conditions such as autism spectrum disorders, social anxiety disorder, or schizophrenia.

17. Social Media Analysis: With the user's consent, the cell phone can access social media data and apply natural language processing and machine learning techniques to analyze the content of posts, comments, or messages. By examining linguistic patterns, sentiment analysis, and social interaction patterns, algorithms can detect potential indicators of mental health disorders, including depression, anxiety, or eating disorders. By accessing the individual's social media accounts and contacts, algorithms can analyze social network data to identify patterns and interactions that may be indicative of mental health disorders. This approach can provide insights into social support networks, social isolation, or dysfunctional social interactions associated with conditions such as social anxiety disorder, borderline personality disorder, or substance use disorders.

18. Speech Pattern Analysis: The cell phone's microphone can be used to analyze speech patterns during the MINI assessment. Advanced speech recognition and natural language processing algorithms can detect linguistic markers, such as speech rate, word choice, or pauses, that may be indicative of mental health disorders like schizophrenia, depression, or cognitive impairments. The cell phone's microphone can be utilized to capture an individual's speech patterns during the MINI assessment. Advanced speech recognition and natural language processing algorithms can analyze speech characteristics, such as speech rate, word choice, and syntactic patterns, to detect linguistic markers associated with specific mental health disorders, including schizophrenia, cognitive disorders, or language impairments.

19. Text Analysis: By analyzing text messages, emails, or other written communication on the cell phone, natural language processing algorithms can detect linguistic markers associated with mental health disorders. This approach can provide insights into cognitive functioning, thought patterns, or emotional states related to conditions such as obsessive-compulsive disorder (OCD), attention-deficit/hyperactivity disorder (ADHD), or personality disorders.

20. Voice Analysis: In addition to capturing speech patterns, the cell phone's microphone can be used for voice analysis during the MINI assessment. Advanced voice recognition algorithms can analyze vocal characteristics, such as pitch, tone, or prosody, to detect markers of mental health disorders, including depression, anxiety, or psychotic disorders. The cell phone's microphone can record an individual's voice during the MINI interview. Voice analysis algorithms can then assess various acoustic features, such as pitch, tone, and speech rate, to detect patterns associated with mental health disorders. This approach can aid in diagnosing conditions like depression, bipolar disorder, or schizophrenia.

Examples Relevant to Beck Depression Inventory (BDI)

1. Activity and Movement Monitoring: The cell phone's accelerometer can be utilized to monitor the individual's activity levels and movement patterns throughout the day. By analyzing changes in physical activity, such as periods of inactivity or excessive restlessness, algorithms can detect potential indicators of depressive symptoms. This approach can provide objective data on the individual's behavioral patterns and activity levels, complementing self-report assessments.

2. Activity and Sleep Monitoring: The cell phone's accelerometer and other sensors can track the individual's activity levels and sleep patterns. Changes in physical activity, sleep duration, or sleep quality can be indicative of depressive symptoms. Advanced algorithms can analyze this data, looking for deviations from the individual's baseline patterns and identifying potential indicators of depression.

3. Contextual Data Integration: The cell phone can collect various contextual data, such as app usage patterns, browsing history, and social media activity. By integrating this data with the BDI self-report assessment, algorithms can analyze how specific digital behaviors and contextual factors correlate with depressive symptoms. For example, excessive use of certain apps or engagement in negative online interactions may indicate depressive tendencies.

4. Environmental Context Monitoring: The cell phone's sensors, including GPS and ambient light sensors, can be used to monitor the individual's environmental context. Algorithms can analyze data such as location, time spent outdoors, exposure to natural light, and changes in environmental factors to assess their potential impact on depressive symptoms. This approach can help identify environmental triggers or protective factors associated with depression.

5. Facial Expression Analysis: The cell phone's camera can be used to capture and analyze facial expressions during the BDI assessment. Advanced facial recognition algorithms can detect subtle changes in facial expressions that may be indicative of depressive symptoms, such as expressions of sadness, hopelessness, or lack of interest. This approach can provide an objective assessment of emotional state and supplement self-report responses. The cell phone's front-facing camera can be used to capture and analyze facial expressions of individuals. Advanced facial expression recognition algorithms can detect subtle changes in facial expressions associated with depressive symptoms, such as expressions of sadness, low mood, or lack of interest. By analyzing facial expressions during self-report assessments or in real-time interactions, this approach can provide additional insights into the individual's emotional state and potential markers of depression.

6. Location and Movement Patterns: The cell phone's GPS capabilities can track the individual's location and movement patterns throughout the day. By analyzing changes in the individual's mobility, frequency of visits to certain locations, or deviations from regular routines, algorithms can identify potential indicators of depressive symptoms. For example, significant changes in the individual's movement patterns, such as increased isolation or avoidance of previously enjoyable activities or places, may suggest depressive symptoms.

7. Location and Social Behavior Tracking: The cell phone's GPS capabilities can be used to track the individual's location and social behavior. By analyzing location patterns, social interactions, and changes in social behavior, algorithms may identify signs of social withdrawal, isolation, or avoidance, which are associated with depressive disorders.

8. Movement and Activity Monitoring: By leveraging the cell phone's accelerometer and gyroscope, algorithms can monitor the individual's movement and activity levels throughout the day. Changes in activity patterns, such as decreased physical activity or increased sedentary behavior, could serve as potential markers of depressive symptoms, as reduced motivation and lack of energy are common features of depression.

9. Real-time Mood Tracking: A dedicated mobile application can be developed to allow individuals to track their mood throughout the day using the BDI criteria. They can provide periodic self-reports of their mood, and the app can prompt them for additional information or context when specific moods are reported. By combining self-reported mood data with other sensor data from the cell phone, such as location or activity levels, algorithms can identify patterns and correlations between mood fluctuations and potential depressive symptoms.

10. Sleep Monitoring: The cell phone's sensors, such as the accelerometer, can be utilized to monitor sleep patterns and disturbances. Disruptions in sleep, such as insomnia or hypersomnia, are frequently observed in individuals with depression. By analyzing sleep data, algorithms could provide additional objective information to support the assessment of depressive symptoms. The cell phone's sensors, such as the accelerometer and ambient light sensor, can be leveraged to monitor sleep patterns and quality. Algorithms can analyze data on sleep duration, sleep efficiency, sleep disturbances, and sleep-wake patterns to assess sleep-related symptoms associated with depression. Sleep disturbances are commonly observed in individuals with depressive disorders, and this approach can provide valuable insights into their sleep patterns and disturbances.

11. Social Interaction Analysis: By analyzing call logs, text message logs, and social media interactions on the cell phone, algorithms can assess the individual's social interaction patterns. Changes in social connectivity, communication frequency, or social engagement levels can be indicators of depressive symptoms, such as social withdrawal or isolation. This approach can provide an objective assessment of the individual's social functioning and social support networks.

12. Social Media Analysis: The cell phone can access the individual's social media accounts and analyze the content and patterns of their posts, comments, and interactions. Natural language processing algorithms can identify linguistic markers associated with depressive symptoms, such as expressions of sadness, hopelessness, or social withdrawal. By examining the individual's social media activity in conjunction with their BDI scores, this approach can provide additional insights into their emotional well-being and help identify potential depressive symptoms. With the individual's consent, access to their social media accounts can be obtained and analyzed using text mining and sentiment analysis techniques. By examining the content of their social media posts, comments, or interactions, algorithms can identify patterns related to depressive symptoms, social withdrawal, or changes in social activity. This approach can provide a broader perspective on the individual's emotional well-being and social functioning.

13. Speech Analysis: In addition to voice analysis, the cell phone's microphone can be utilized to analyze speech patterns and language use during the BDI assessment. Natural language processing algorithms can identify linguistic markers associated with depressive symptoms, such as negative word usage, low self-esteem, or cognitive distortions. This approach can provide insights into the individual's cognitive and emotional processes.

14. Speech Pattern Analysis: In addition to voice analysis, more comprehensive speech pattern analysis can be conducted using the cell phone's microphone. Advanced natural language processing algorithms can analyze speech content, syntax, and semantic features to identify linguistic markers of depressive symptoms. Changes in speech patterns, such as increased use of negative language, reduced cognitive complexity, or alterations in speech fluency, can provide additional insights into the individual's mental health.

15. Voice Analysis: The cell phone's microphone can be used to analyze voice characteristics and patterns during self-report assessments or phone conversations. Advanced voice analysis algorithms can detect variations in speech features, such as pitch, tone, rhythm, and speech rate, which may be associated with depressive symptoms. This approach can provide insights into the individual's emotional state and potential markers of depression. The cell phone's microphone can be used to analyze the individual's voice and detect changes associated with depressive symptoms. Voice analysis algorithms can assess various acoustic features, such as pitch, tone, and speech rate, to identify vocal characteristics that may indicate depression. By comparing the individual's voice patterns with their BDI scores, this approach can provide additional objective measures of their mental health. The cell phone's microphone can be used to capture and analyze the individual's voice during the BDI assessment. Advanced voice analysis algorithms can detect changes in pitch, tone, or speech patterns that may be indicative of depressive symptoms. This approach could complement the self-report responses and provide additional insights into the individual's emotional state.

Examples Relevant to Hamilton Rating Scale for Anxiety (HAM-A)

1. Activity and Movement Tracking: The cell phone's accelerometer and GPS can track the individual's activity levels and movement patterns. Increased restlessness, fidgeting, or changes in movement patterns can be indicative of anxiety. Advanced algorithms can analyze this data, looking for deviations from the individual's baseline patterns and identifying potential indicators of anxiety.

The cell phone's accelerometer can capture motion data and detect physical activity levels throughout the day. Algorithms can analyze activity patterns, such as increased restlessness or fidgeting, which may be associated with anxiety. By correlating these activity measures with the HAM-A criteria, it becomes possible to gain insights into the relationship between physical agitation and anxiety symptom severity.

2. Activity Monitoring: The cell phone's accelerometer can track an individual's activity levels and movements throughout the day. By analyzing changes in physical activity, restlessness, or patterns of sedentary behavior, algorithms can detect potential indicators of anxiety. These activity patterns can be correlated with the HAM-A criteria to provide insights into the relationship between physical behavior and anxiety symptoms.

3. Contextual Data Analysis: The cell phone can collect contextual data, such as location, time of day, social interactions, and communication patterns. By integrating this data with the individual's HAM-A scores, algorithms can identify correlations between specific contexts or triggers and anxiety levels. This information can help in understanding the environmental and situational factors that contribute to anxiety symptoms. In addition to utilizing individual cell phone features, integrating contextual data from various sources can provide a more comprehensive understanding of anxiety disorders. This may include integrating data from wearable devices, such as heart rate monitors or sleep trackers, along with cell phone data. By combining physiological measures, behavioral patterns, and environmental factors, algorithms can generate personalized anxiety profiles aligned with the HAM-A criteria.

4. Environmental Contextual Analysis: By leveraging the cell phone's sensors, including the microphone and GPS, the environmental context surrounding an individual can be analyzed. This analysis can include factors such as ambient noise levels, proximity to crowded places, or exposure to triggering environments. By integrating this contextual information with the HAM-A criteria, a more comprehensive understanding of anxiety triggers and symptom exacerbation can be obtained.

5. Environmental Noise Monitoring: The cell phone's microphone can be utilized to monitor the ambient noise levels in an individual's environment. Excessive noise or specific sound patterns may contribute to anxiety symptoms. Algorithms can analyze noise levels, frequency spectra, and specific acoustic signatures to identify environments that may trigger anxiety. By integrating this information with the HAM-A criteria, a more comprehensive assessment of anxiety-related environmental factors can be achieved.

6. Facial Expression Analysis: The cell phone's camera can be used to capture facial expressions and analyze them for signs of anxiety. Facial expression recognition algorithms can detect subtle changes in facial muscle movements and provide objective measures of anxiety levels. This analysis can be performed in real-time or through recorded video clips, allowing for continuous monitoring and assessment of anxiety symptoms. By detecting facial features and expressions associated with anxiety, such as increased muscle tension, furrowed brows, or tense jaw, algorithms can assess anxiety symptom severity. Real-time or recorded video analysis can provide objective data for evaluating anxiety-related facial expressions in line with the HAM-A criteria. The cell phone's camera can capture the individual's facial expressions, and advanced facial recognition algorithms can analyze these images to identify potential signs of anxiety. Facial expression analysis can detect subtle changes in facial muscle movements, such as furrowed brows, tense jaw, or rapid eye movements, which are associated with anxiety. By comparing the captured facial expressions with the HAM-A criteria, this approach can provide objective measures of anxiety levels.

7. Heart Rate Variability Analysis: The cell phone's camera can be used in conjunction with specialized sensors to measure heart rate variability (HRV), which refers to the variation in time intervals between consecutive heartbeats. HRV analysis has shown promise in assessing anxiety levels. By combining the cell phone's camera with a compatible HRV sensor, real-time measurements can be obtained, and the data can be analyzed to identify patterns associated with anxiety.

8. Location-Based Triggers: By utilizing the cell phone's GPS functionality, location-based triggers can be identified and linked to anxiety symptoms. For example, if an individual consistently experiences higher anxiety levels in specific locations, such as crowded places or certain social environments, the cell phone can detect these patterns. This information can be correlated with the HAM-A criteria to provide insights into the contextual factors contributing to anxiety. The cell phone's GPS can be used to track an individual's location and identify specific places or situations that trigger anxiety symptoms. Through geolocation data and user input, algorithms can recognize patterns of anxiety-inducing locations and provide personalized insights into the environmental triggers. This information can complement the HAM-A criteria to better understand the context-specific nature of anxiety symptoms.

9. Multimodal Data Fusion: By combining data from various sensors on the cell phone, such as the camera, microphone, accelerometer, and GPS, along with additional contextual information, a comprehensive and multimodal analysis can be performed. Machine learning algorithms can integrate and analyze these diverse data sources to identify complex patterns and relationships associated with anxiety disorders. This holistic approach can provide a more accurate and personalized assessment of anxiety levels based on the HAM-A criteria. Combining data from multiple cell phone sensors, such as the camera, microphone, accelerometer, and GPS, along with other contextual information, allows for a more comprehensive assessment of anxiety disorders. By integrating data from various sources and applying machine learning techniques, algorithms can identify complex patterns and relationships between sensor data, behavior, and anxiety symptoms. This multimodal data fusion approach offers a holistic perspective on mental health assessment.

10. Sleep Monitoring: Many cell phones have built-in sleep tracking capabilities or can be paired with wearable devices to monitor sleep patterns. By analyzing sleep duration, sleep quality, and disruptions during sleep, algorithms can identify sleep disturbances commonly associated with anxiety disorders. Sleep-related data can be integrated with the HAM-A criteria to assess the impact of anxiety on sleep and overall symptomatology. The cell phone's accelerometer can be used to monitor an individual's sleep patterns and detect potential sleep disturbances associated with anxiety disorders. Sleep quality, duration, and disruptions can be analyzed to identify correlations with anxiety symptoms. Advanced algorithms can process the accelerometer data to provide objective measures of sleep quality and identify patterns that may indicate anxiety-related sleep disturbances.

11. Social Interaction Analysis: By leveraging the cell phone's communication features, such as call logs, text messages, and social media interactions, algorithms can analyze the frequency and quality of social interactions. An individual's social network dynamics, patterns of communication, and changes in social engagement can be correlated with the HAM-A criteria to assess the impact of social factors on anxiety levels and symptom severity. The cell phone's microphone and text analysis capabilities can be utilized to analyze social interactions. Speech patterns, tone of voice, and language use during conversations or phone calls can provide valuable information about an individual's anxiety levels. By comparing the analyzed social interaction data with the HAM-A criteria, specific anxiety-related patterns can be identified.

12. Social Media Analysis: With the user's consent, data from social media platforms can be integrated with the HAM-A criteria to identify anxiety-related patterns. Natural language processing techniques can analyze the content of social media posts, comments, and interactions to detect signs of anxiety and correlate them with the HAM-A criteria. This approach provides a unique insight into an individual's mental state and social interactions outside of clinical settings.

13. Speech Analysis: The cell phone's microphone can be utilized to analyze speech patterns and linguistic features in real-time conversations or recorded audio. Natural language processing algorithms can identify specific speech characteristics associated with anxiety, such as increased speech rate, use of specific words or phrases, or speech hesitation. By comparing these patterns with the HAM-A criteria, it can provide insights into anxiety symptom severity and fluctuations.

14. Text Analysis: The cell phone's text messaging or chat applications can be analyzed using natural language processing algorithms to identify linguistic markers associated with anxiety. This analysis can detect keywords, phrases, or patterns indicative of anxious thoughts or behaviors. By comparing the individual's text messages with the HAM-A criteria, this approach can provide additional insights into their anxiety levels and thought patterns.

15. Voice Analysis: The cell phone's microphone can be used to analyze the individual's voice and detect changes associated with anxiety. Voice analysis algorithms can assess various acoustic features, such as pitch, speech rate, and intonation, to identify vocal characteristics that may indicate anxiety. By comparing the individual's voice patterns with the HAM-A scores, this approach can provide additional insights into their anxiety levels. By capturing and analyzing vocal characteristics, such as pitch, intensity, and speech rate, algorithms can identify specific markers associated with anxiety. This voice analysis can be combined with the HAM-A criteria to provide objective measurements of anxiety levels.

16. Voice Modulation Analysis: The cell phone's microphone can be employed to analyze voice modulation and acoustic features during conversations or recordings. Changes in voice pitch, volume, and speech patterns can be indicative of anxiety symptoms. By using signal processing and machine learning algorithms, these acoustic features can be quantified and correlated with the HAM-A criteria to assess anxiety severity and symptomatology.

Examples Relevant to Yale-Brown Obsessive-Compulsive Scale (Y-BOCS)

1. Ambient Audio Analysis for Vocal or Mental Rituals: The cell phone's microphone can be employed to capture ambient audio and analyze it for vocal or mental rituals. By analyzing audio recordings for repetitive vocalizations or mental rituals, algorithms can assess the presence and severity of OCD symptoms according to the Y-BOCS criteria.

2. Audio Recording and Analysis: The cell phone's microphone can record audio in situations where obsessive thoughts or compulsive rituals occur. Advanced speech and audio analysis algorithms can be applied to detect specific linguistic patterns, such as repeated phrases or vocalizations, that are indicative of obsessive thoughts or compulsive behaviors. By comparing these audio recordings with the Y-BOCS criteria, clinicians can gain a deeper understanding of symptomatology and severity.

3. Behavior Monitoring and Tracking: The cell phone's accelerometer and GPS can be used to monitor and track an individual's movements and behaviors. Algorithms can analyze patterns of repetitive behaviors, such as handwashing, checking, or arranging objects, by detecting specific movement patterns and locations. By comparing these behavioral patterns with the Y-BOCS criteria, it becomes possible to assess the severity and frequency of obsessive-compulsive symptoms.

4. Combined Sensor Data Fusion: Integrating data from multiple sensors on the cell phone, such as the camera, microphone, accelerometer, and GPS, can provide a more comprehensive assessment of OCD symptoms. Algorithms can analyze the combined data to identify correlations, patterns, and triggers related to obsessions and compulsions, aiding in diagnosis and treatment planning.

5. Communication Analysis for Seeking Reassurance: The cell phone's communication features, such as calls, messages, and social media interactions, can be analyzed to detect patterns of seeking reassurance. By examining communication content and frequency, algorithms can identify instances where individuals with OCD seek reassurance from others, providing valuable information for diagnosis and treatment planning.

6. Contextual Data Integration: Integrating data from multiple sources, including the cell phone's features and other wearable devices, can provide a more comprehensive understanding of obsessive-compulsive disorder (OCD). This integration can include data on physiological measures (e.g., heart rate variability), sleep patterns, location data, and social interactions. By combining these various data sources and aligning them with the Y-BOCS criteria, clinicians can develop a more holistic view of OCD symptoms and triggers.

7. Geolocation Tracking for Avoidance Behaviors: The cell phone's GPS capabilities can be utilized to track an individual's location and identify patterns of avoidance behaviors associated with obsessive-compulsive symptoms. By analyzing movement patterns and identifying specific locations or situations avoided due to obsessions or compulsions, clinicians can assess the impact of avoidance behaviors on an individual's life.

8. Image and Object Recognition: The cell phone's camera can be utilized to capture images of objects or situations that trigger obsessive or compulsive behaviors. By using computer vision algorithms, these images can be analyzed to detect specific objects or visual cues associated with obsessions or compulsions. This analysis can provide insights into the relationship between triggers and symptom severity based on the Y-BOCS criteria.

9. Image Recognition for Compulsive Checking Behaviors: The cell phone's camera can be used in combination with image recognition algorithms to detect and analyze compulsive checking behaviors. By capturing and analyzing images of repeatedly checked objects or locations, the algorithm can identify patterns of compulsive behavior and assess their severity based on the Y-BOCS criteria.

10. Image Recognition for Compulsive Checking: The cell phone's camera can be utilized to analyze images captured by individuals with obsessive-compulsive checking behaviors. By applying image recognition algorithms, specific objects or areas that trigger checking behaviors can be identified, providing insights into the severity and frequency of compulsions.

11. Keyboard Interaction Analysis: The cell phone's touch-screen keyboard can be used to analyze typing behavior and patterns. Algorithms can detect repetitive or excessive typing, specific word choices, or patterns of backspacing and correction that may indicate obsessive or compulsive thoughts. By comparing these typing patterns with the Y-BOCS criteria, it becomes possible to assess the presence and severity of obsessive-compulsive symptoms.

12. Location Tracking for Ritualistic Behaviors: The cell phone's GPS capability can be utilized to track an individual's location and identify patterns related to ritualistic behaviors. By analyzing location data and correlating it with specific rituals or compulsions, algorithms can provide insights into the environmental triggers and contexts associated with obsessive-compulsive symptoms.

13. Machine Learning-Based Models for Symptom Prediction: By collecting data from various sensors on the cell phone, such as the camera, microphone, accelerometer, and GPS, machine learning algorithms can be trained to predict OCD symptoms based on patterns and correlations in the data. These predictive models can assist in early detection, monitoring, and intervention for individuals at risk or already diagnosed with OCD.

14. Machine Learning-based Symptom Prediction: By collecting extensive data from cell phone sensors, self-reporting tools, and other digital sources over time, machine learning algorithms can be trained to predict the occurrence and severity of obsessive-compulsive symptoms. These predictive models can identify early warning signs, triggers, or contextual factors that contribute to symptom exacerbation. By providing personalized insights, this approach can support proactive interventions and self-management of OCD.

15. Movement Analysis for Motor Compulsions: The cell phone's accelerometer can be employed to detect and analyze repetitive movements associated with motor compulsions. By monitoring movement patterns and frequencies, algorithms can identify specific motor actions performed repeatedly, assisting in the assessment of compulsive behaviors.

16. Movement Tracking and Accelerometer Data: The cell phone's accelerometer can capture movement patterns and data related to compulsive rituals. By analyzing the intensity, frequency, and duration of repetitive movements, algorithms can provide insights into the severity of compulsions and their impact on daily functioning.

17. Real-Time Symptom Tracking: Utilizing mobile applications, individuals can self-report their obsessive thoughts, compulsive behaviors, and associated distress in real-time using their cell phones. These self-reporting tools can include validated questionnaires and prompts that align with the Y-BOCS criteria. By capturing real-time symptom data, clinicians can gain insights into the temporal patterns, triggers, and fluctuations of OCD symptoms.

18. Sensor Data Fusion: By combining multiple sensors in a cell phone, such as the camera, microphone, accelerometer, and GPS, it is possible to create a holistic view of an individual's behaviors and experiences related to OCD. Data from these sensors can be fused and analyzed using machine learning algorithms to identify unique patterns, triggers, and contexts associated with obsessive-compulsive symptoms. This approach can provide a comprehensive assessment of an individual's symptom severity and guide treatment strategies.

19. Smartphone-based Ecological Momentary Assessment (EMA): EMA involves collecting real-time data on an individual's thoughts, feelings, and behaviors in their natural environment. Through smartphone apps, individuals can provide self-reports at specific intervals, allowing for the assessment of OCD symptoms based on the Y-BOCS criteria in real-time and in various contexts.

20. Social Media Analysis: Social media platforms can be integrated with the Y-BOCS criteria to identify patterns of behavior and content related to obsessive-compulsive symptoms. Natural language processing algorithms can analyze social media posts, comments, and interactions to detect signs of obsessive thoughts, compulsive behaviors, or related distress. This analysis can provide additional insights into an individual's symptomatology and help clinicians assess the impact of OCD on their daily life.

21. Text and Language Analysis for Intrusive Thoughts: The cell phone's text messaging and keyboard input can be used to analyze the content and frequency of intrusive thoughts associated with OCD. Natural language processing algorithms can identify specific keywords, themes, or linguistic patterns related to obsessions, providing insights into the severity and nature of intrusive thoughts.

22. Touchscreen Interactions for Compulsive Tapping or Touching: The touchscreen interface of a cell phone can be utilized to track and analyze compulsive tapping or touching behaviors. By monitoring touch patterns and durations, algorithms can identify excessive and repetitive tapping or touching behaviors associated with obsessive-compulsive symptoms.

23. User Interaction Data for Symptom Tracking: Cell phone usage data, including app usage, screen time, and interaction patterns, can be analyzed to track OCD symptoms over time. By examining the frequency and duration of specific behaviors, such as checking or organizing apps, algorithms can provide objective measures of symptom severity and progression.

24. Virtual Reality Exposures: Virtual reality (VR) technologies can be integrated with the Y-BOCS criteria to create immersive and controlled environments for exposure therapy under supervision by a licensed mental health provider. Using a cell phone and VR headset, individuals can be exposed to virtual scenarios that trigger their obsessive thoughts or compulsive behaviors. The cell phone's sensors can track physiological responses, movement, and interactions within the virtual environment, providing real-time data to assess symptom responses and guide treatment progress.

25. Voice Analysis for Obsessive Thoughts: The cell phone's microphone can be utilized to record and analyze an individual's voice patterns for signs of obsessive thoughts. Natural language processing algorithms can detect specific keywords, repetitions, or other linguistic patterns associated with obsessions, providing an objective measure for assessing symptom severity.

Examples Relevant to Brief Psychiatric Rating Scale (BPRS)

1. Activity Monitoring for Psychomotor Symptoms: The cell phone's accelerometer and gyroscope can be employed to monitor an individual's movement and activity levels. By analyzing activity patterns, algorithms can detect and quantify psychomotor symptoms, such as agitation or retardation, which are important indicators for various mental health disorders. This data can assist in diagnosis, treatment monitoring, and personalized interventions.

2. Contextual Data Collection: The cell phone's sensors, including the camera, microphone, and GPS, can be used to collect contextual data related to an individual's daily activities and environment. This data can provide valuable information about the individual's routines, social interactions, environmental triggers, and overall functioning. Integrating this data with BPRS assessments can offer a more comprehensive understanding of the individual's mental health status.

3. Digital Diary for Symptom Tracking: A cell phone application can be developed to allow individuals to self-report symptoms using text, voice recordings, or multimedia elements. By implementing structured prompts and questionnaires aligned with the BPRS criteria, this digital diary can capture real-time symptom experiences, including mood changes, perceptual disturbances, or changes in behavior. This data can be analyzed to identify trends, triggers, and severity of symptoms.

4. Digital Phenotyping for Symptom Clusters: By leveraging multiple sensors on the cell phone, such as the camera, microphone, accelerometer, and GPS, comprehensive data can be collected for digital phenotyping. Machine learning algorithms can then analyze this data to identify patterns, correlations, and clusters of symptoms associated with various mental health disorders. This approach can assist in diagnosing and understanding complex symptom presentations.

5. Ecological Momentary Assessment (EMA): The cell phone can be used for real-time data collection through EMA methods. Individuals can receive prompts on their cell phones to provide self-assessments of their symptoms and experiences multiple times a day. This allows for capturing momentary changes in symptoms, triggers, and stressors, providing a more nuanced understanding of the individual's mental health state.

6. Environmental Noise Monitoring for Anxiety and Stress: The cell phone's microphone can be used to monitor ambient noise levels in the environment. Machine learning algorithms can analyze the audio data to detect and quantify noise levels associated with anxiety and stress. High levels of noise may trigger or exacerbate symptoms in individuals with anxiety disorders, and this information can be used to understand environmental triggers.

7. Facial Expression Analysis: The cell phone's camera can be used to capture facial expressions and analyze them using computer vision and facial recognition algorithms. By detecting facial cues associated with different mental health disorders, such as expressions of sadness, fear, or agitation, the phone can provide objective measures for assessing symptom severity and tracking changes over time.

8. Facial Expression Recognition for Emotional Disturbances: The cell phone's camera can be used to capture and analyze facial expressions in real-time. By applying facial expression recognition algorithms, the phone can detect and assess emotional disturbances associated with mental health disorders, such as flat affect or exaggerated expressions. This technology can provide objective measures of emotional states and aid in diagnosis and treatment planning.

9. Location and Movement Analysis for Paranoia and Delusions: The cell phone's GPS and movement sensors can be used to track an individual's location and movement patterns. By analyzing this data alongside self-reported experiences, algorithms can detect potential patterns related to paranoia and delusions. For example, sudden changes in movement or frequent visits to specific locations may indicate heightened suspiciousness. This information can contribute to the assessment and understanding of psychotic symptoms.

10. Location-Based Triggers: By utilizing the cell phone's GPS capabilities, location data can be integrated into the assessment of mental health disorders. The phone can track an individual's movements and identify specific locations or environments that trigger symptoms or distress. This information can help clinicians understand the impact of environmental factors on mental health and develop targeted interventions.

11. Mood and Emotion Tracking: Mobile apps can be developed to allow individuals to self-report their mood and emotions using standardized scales based on BPRS criteria. The app can prompt users to rate their mood throughout the day, and additional features like voice recording or facial expression analysis can be integrated for more objective assessment. Machine learning algorithms can analyze the data to detect patterns and provide insights into mood fluctuations and symptom severity.

12. Movement and Motor Behavior Monitoring: The cell phone's accelerometer and gyroscope can be used to monitor an individual's movement patterns and motor behavior. Changes in motor activity, such as psychomotor agitation or retardation, can be indicative of certain mental health disorders. Machine learning algorithms can analyze the sensor data to identify abnormal movement patterns and provide objective measures for assessment.

13. Sleep Monitoring for Psychopathology: The cell phone's accelerometer and gyroscope can be utilized to monitor sleep patterns and disturbances. Algorithms can analyze movement data during sleep to detect insomnia symptoms, sleep fragmentation, or abnormal sleep behaviors associated with mental health disorders. This information can contribute to the assessment of sleep-related psychopathology.

14. Sleep Monitoring: The cell phone's accelerometer can be used to track an individual's sleep patterns, including sleep duration, quality, and disruptions. Disrupted sleep is often associated with various mental health disorders, such as anxiety, depression, or bipolar disorder. By analyzing the accelerometer data, sleep disturbances can be identified and correlated with symptom severity, aiding in the diagnostic process.

15. Social Interaction Analysis: The cell phone can analyze social interaction patterns by monitoring call logs, text messages, or social media interactions. Machine learning algorithms can identify social withdrawal, reduced communication, or changes in social network dynamics, which can be indicative of social anxiety, depression, or other mental health disorders.

16. Social Media Analysis for Mood and Behavior Assessment: By integrating with social media platforms, the cell phone can collect data on an individual's online behavior, content, and interactions. Natural language processing and sentiment analysis techniques can be employed to assess mood states, identify depressive or manic symptoms, detect changes in social interaction patterns, or identify potential suicidal ideation or self-harm risks.

17. Speech Analysis for Thought Disorders: The cell phone's microphone can be utilized to analyze speech patterns and content for signs of thought disorders associated with mental health disorders. Natural language processing algorithms can detect features such as tangential or disorganized speech, word salad, or derailment, providing objective measures to aid in diagnosing conditions like schizophrenia or psychotic disorders.

18. Speech Analysis: The cell phone's microphone can be used to record and analyze an individual's speech patterns, including speech rate, fluency, and content. Natural language processing algorithms can be employed to detect linguistic markers associated with different mental health disorders. Changes in speech patterns, such as disorganized or pressured speech, can provide valuable insights for diagnosis and monitoring treatment progress.

19. Voice Analysis for Speech Patterns: The cell phone's microphone can be utilized to analyze speech patterns and characteristics, such as tone, pace, and content. By employing voice analysis algorithms, deviations from normal speech patterns associated with mental health disorders, such as disorganized or pressured speech, can be identified and quantified, providing valuable insights for diagnosis and monitoring.

20. Voice Modulation Analysis: The cell phone's microphone can be used to capture and analyze variations in voice modulation, pitch, and tone. These acoustic features can provide valuable insights into mood fluctuations, emotional instability, or signs of psychotic symptoms. Machine learning algorithms can be trained to recognize patterns associated with different mental health disorders, assisting in diagnosis and treatment monitoring.

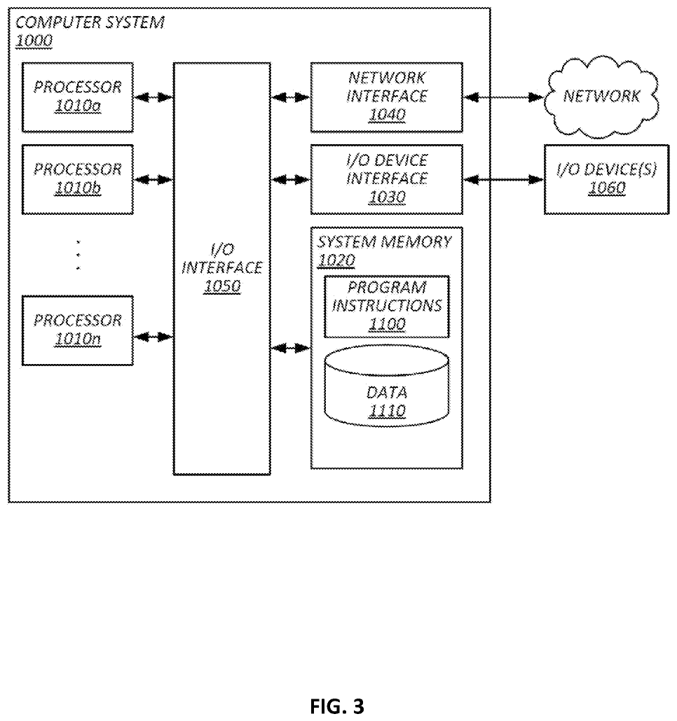

FIG. 3 is a diagram that illustrates an exemplary computing system 1000 in accordance with embodiments of the present technique. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer systems, for example, may be connected to computer system 1000 via a network and network interface 1040.

Network interface 1040 may include a network adapter that provides for connection of computer system 1000 to a network. Network interface 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to cost constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

What is claimed is:

1. A tangible, non-transitory, machine-readable medium storing instructions that when executed by one or more processors effectuate operations comprising:
obtaining data from a sensor or user-interface of a mobile computing device gathered during use of the mobile computing device by a user;
inferring, from the data, with a trained machine learning model, a mental-health state of the user; and
storing the mental health state in memory.

2. The medium of claim 1, wherein:
the data includes a behavioral pattern or digital biomarker of the user; and
at least some of the data is gathered responsive to the user granting permission to do so.

3. The medium of claim 1, wherein the data include:
usage of an application installed on the mobile computing device;
typing and touchscreen input variability of the user on the mobile computing device;
scrolling behavior of the user on the mobile computing device; or
typing errors of the user on the mobile computing device.

4. The medium of claim 1, wherein:
the data include:
usage of an application installed on the mobile computing device;
typing and touchscreen input variability of the user on the mobile computing device;
scrolling behavior of the user on the mobile computing device; and
typing errors of the user on the mobile computing device, and wherein:
the data is multimodal; and
the machine learning model includes a neural network with more than three layers configured to classify whether the data is indicative of a mental health disorder.

5. The medium of claim 4, wherein:
the machine learning model comprises an anomaly detection model configured to detect deviation from baseline data of the user or a population of users.

6. The medium of claim 1, wherein:
the data indicates which applications installed on the mobile computing device are used and amounts of usage of each such application used; and
the machine learning model is responsive to changes in relative amounts of usage of different categories of applications, the different categories including productivity applications and social media applications.

7. The medium of claim 1, wherein:
the data includes speed, frequency, and direction of scrolling, by the user, of one or more user interfaces displayed by the mobile computing device; and
the machine learning model is configured to detect anomalous patterns in the data indicative of the mental-health state of the user.

8. The medium of claim 1, wherein:
the machine learning model is configured to determine a score based on an amount of switching between tasks by the user on the mobile computing device and determine whether the score satisfies a threshold associated with a mental health condition.

9. The medium of claim 1, wherein:
the machine learning model is configured to infer the mental-health state of the user based on patterns of usage throughout a 24-hour cycle, indicative of sleep disturbances or disruptions.

10. The medium of claim 1, wherein:
the data includes text input by the user; and
the machine learning model includes a natural language processing model configured to infer the mental-health state of the user based on the text input by the user.

11. The medium of claim 1, wherein:
the data includes an image of a face or body of the user captured by a camera of the mobile computing device; and
the machine-learning model comprises a computer vision model configured to infer the mental-health state of the user based on facial expression analysis of the image.

12. The medium of claim 11, wherein:
the computer vision model is configured to provide real-time inference of the mental-health state of the user within five seconds of capturing the image with the camera; and
the computer vision model is configured to detect facial landmarks associated with different emotions.

13. The medium of claim 11, wherein:
the image is a frame of video captured by a the camera; and
the computer vision model is configured to infer the mental health state of the user based on movement detected based on differences between frames of the video.

14. The medium of claim 11, wherein:
the computer vision model is a deep learning model trained by obtaining a training set with more than 500 images of faces and learning to perform automated feature extraction for features corresponding to facial expression changes with emotion changes based on the training set.

15. The medium of claim 11, wherein the operations comprise:
assessing changes in emotional state of the user over time based on intensity and duration of emotional states inferred from a plurality of images captured over time, the plurality of images including the image.

16. The medium of claim 1, wherein:
the data comprise responses obtained by prompting the user to provide self-reported mood states or by inferring mood states of the user over time.

17. The medium of claim 16, wherein:
the machine learning model is configured to infer triggers of, or patterns in, changes in the mental health state based on the data.

18. The medium of claim 16, wherein:
the data comprises inferred mood states; and
the mood states are inferred based on user location, activity level, social interactions, or physiological measurements of the user.

19. The medium of claim 18, wherein:
the mood states are inferred based on user location, activity level, social interactions, and physiological measurements of the user; and
the physiological measurements of the user include heart rate and sleep quality.

20. The medium of claim 16, wherein the operations comprise:
recommending an intervention to the user based on inferences from the machine learning model.

21. The medium of claim 1, wherein:
the data comprises multiple channels of data from multiple sensors of the mobile computing device, the sensors including an inertial measurement device having three or more axes, a geolocation sensor, a microphone, and a heart rate sensor.

22. The medium of claim 21, wherein:
output from the accelerometer is used to form features used by the machine learning model indicative of physical activity, gestures, and behavioral patterns of the user.

23. The medium of claim 21, wherein:
the geolocation sensor is a satellite navigation sensor;
output from the geolocation sensor is used to form features used by the machine learning model indicative of mobility patterns, travel habits, and exposure to different environments.

24. The medium of claim 21, wherein:
output from the microphone is used to form features used by the machine learning model indicative of speech patterns, social interactions, and ambient sounds.

25. The medium of claim 21, wherein:
output from the heart rate sensor, and variability thereof over time, is used to form features used by the machine learning model indicative of physiological arousal, stress levels, and emotional states of the user.

26. The medium of claim 21, the operations further comprising:
determining a personalized intervention based on the inferred mental-health state of the user.

27. The medium of claim 1, wherein:
the machine learning model is configured to perform active learning.

28. A method, comprising:
obtaining data from a sensor or user-interface of a mobile computing device gathered during use of the mobile computing device by a user;
inferring, from the data, with a trained machine learning model, a mental-health state of the user; and
storing the mental health state in memory.
